(19) United States
(12) Patent Application Publication
(10) Pub. No.: US 2008/0312102 A1
(43) Pub. Date: Dec. 18, 2008

(54) Cytokine Binding Domains

(75) Inventors: Joseph Noozhumutry Varghese, Victoria (AU); Peter John Hudson, Victoria (AU); Barbara Elaine Power, Victoria (AU)

Correspondence Address: BOZICEVIC, FIELD & FRANCIS LLP, 1900 UNIVERSITY AVENUE, SUITE 200, EAST PALO ALTO, CA, 94303, US

(73) Assignee: COMMONWEALTH SCIENTIFIC AND INDUSTRIAL RESEARCH ORGANISATION, Campbell (AU)

(21) Appl. No.: 10/582,951

(22) PCT Filed: Dec. 16, 2004
(86) PCT No.: PCT/AU2004/001762

§ 371 Date: Jun. 18, 2008

(30) Foreign Application Priority Data
Dec. 16, 2003 (AU) 2003906979

(51) Int. Cl. C40B 40/08; C07K 2/00; G01N 33/566; A61K 38/02; C07K 14/47

(52) U.S. Cl. 506/17; 530/300; 530/350; 436/501; 514/2

(57) ABSTRACT

The present invention relates a method of generating modified binding moieties comprising a cytokine binding domain consisting of a first FmIII-like domain and a second FnIII-like domain in which at least one amino acid residue within the cytokine binding domain in modified such that the binding characteristics of the cytokine binding domain are altered, and/or, the solubility and/or stability of the binding moiety is improved. The invention also relates to binding moieties and to the use of such binding moieties as affinity reagents, diagnostic reagents, therapeutic agents and protein scaffolds

### FIELD OF THE INVENTION

The present invention relates to binding moieties derived from cytokine binding domains (CBDs) and their use as affinity reagents, diagnostic reagents, therapeutic agents and as protein scaffolds.

### BACKGROUND TO THE INVENTION

Antibodies are the paradigm of specific high-affinity binding reagents and provide an antigen binding site by interaction of variable heavy (V_{H}) and variable light (V_{L}) immunoglobulin domains. The binding interface is formed by six surface polypeptide loops, termed complementarity determining regions (CDRs), three from each variable domain, which are highly variable and combined provide a sufficiently large surface area for interaction with antigen. Specific binding reagents can be formed by association of only the V_{H} and V_{L} domains into an Fv module. Bacterial expression is enhanced by joining the V-domains with a linker polypeptide into a single-chain scFv molecule.

WO 00/34784 and WO 01/64942 (Phylos Inc.) disclose antibody mimics comprising a fibronectin or fibronectin-like protein scaffold in which a fibronectin type III domain having at least one randomised loop is present. WO 02/32925 (Phylos Inc.) relates to non-antibody derivative proteins comprising a domain having an immunoglobulin-like fold in which the protein has a mutated amino acid sequence such that its binds to a compound with greater affinity than the unmutated protein.

Koide et al. (WO 98/56915 and J. Mol. Biol., (1998), 284, 1141-1151) describe the design and construction of a fibronectin type III domain scaffold and the use of the scaffold to produce a phage display library with mutation in two loops to screen for higher affinity ligand binding.

WO 01/90192 (Imclone Systems Inc) describes a bispecific two-chain immunoglobulin construct, a two domain protein which is optimized in its avidity for antigen but still acts as a natural antibody.

WO 02/48189 (Borean Pharma AS) describes a scaffold based on the family of C-type lectin-like domains, which has a carbohydrate recognition domain having a loop region that can be mutated so as to provide a new class of libraries.

WO 00/47620 (Medvet Science Pty Ltd et al.) discloses a cytokine-binding domain that consists of a β-chain or analogous structure of a cytokine receptor.

WO 02/44197 (Fish) describes cytokine receptor binding peptide constructs in which the cytokine receptor binding domain is incorporated into a scaffold such that the scaffold maintains the binding domain configuration suitable for binding to the cytokine receptor.

### SUMMARY OF THE INVENTION

The present invention relates to binding moieties which employ a CBD-like scaffold structure consisting of two FnIII-like domains as schematically depicted in FIG. 1A. Solvent exposed loops on the two FnIII-like domains are in linear association and define a binding region which is capable of binding to a target molecule through association with loops from both domains. The invention also relates to a method for producing novel scaffold structures based on the use of cytokine-binding domains (CBDs) as well as the novel scaffold structures produced thereby.

Accordingly, the invention provides to a method of producing a binding moiety comprising modifying an extracellular cytokine binding domain consisting of a first FnIII-like domain and a second FnIII-like domain such that at least one property of the cytokine binding domain is altered, to produce a binding moiety.

Furthermore, the invention provides a modified binding moiety produced according to the above method of the invention.

The present invention also provides novel binding moieties based on a cytokine binding domain scaffold structure.

Accordingly, the invention also provides a binding moiety comprising an extracellular cytokine binding domain consisting of a first FnIII-like domain and a second FnIII-like domain, wherein the CBD comprises a modification which alters a property of the CBD.

CBDs consist of two linked fibronectin type III (FnIII) domains (each an Ig-like fold) (Leahy D J et al., 1992, Science 258: 987-991). These CBDs are known to bind their target molecules primarily at the juncture of the two FnIII-like domains (the cytokine hinging region), engaging their target molecules by loops on the outer elbow of the two domains of the CBD. These loops are similar to the CDR (complementarity determining region) loops found on the antigen-binding surface of antibody variable domains. However, the association between loops from the two domains in a CBD exhibits important differences to antibody CDR loop association. In antibody variable domains, the loops from the heavy chain associate in parallel with those of the light chain. In contrast, the cytokine binding loops of cytokine binding regions form a linear association (see FIG. 1). A comparison between the CBDs of a number of know tertiary structures reveal common structural features indicating that these domains form an ideal framework for designing and generating novel binding moieties. Such binding moieties will have a variety of uses and applications including, as diagnostic and therapeutic agents/reagents, being directed to particular molecular targets, and in particular those targets associated with clinical disease.

The prior art typically describes scaffold structures based on single binding domains. In particular, previous work on scaffolds utilising FnIII-like domains has concentrated on the use of single FnIII-like domain frameworks. In contrast, the scaffolds of the invention are based on the use of CBDs having two FnIII-like domains, in which a target molecule can be bound through association with both domains, and more particularly through interaction with loops forming the cytokine binding region of the CBD.

The scaffolds of the invention provide significant advantages over the prior art scaffolds. The use of a two-domain binding moiety results in a larger surface binding area or “footprint” for binding with a target molecule. This creates the potential for binding with higher affinity and/or to a greater variety of target shapes and sizes. In particular, the use of a two-domain, linearly associated framework creates the potential for these moieties to bind to molecules that are refractory to conventional antibodies.

The binding moieties of the invention may be linked to other molecules, for example by covalent or non-covalent means. Accordingly, the invention provides a binding moiety according to the invention linked to one or more other molecules.

Furthermore, the invention provides a multivalent or multispecific reagent comprising two or more binding moieties according to the invention.

The invention also provides a polynucleotide encoding a binding moiety, multivalent reagent or multispecific reagent according to the invention.

The invention also provides a vector comprising a polynucleotide according to the invention.

The invention also provides a host cell comprising a vector according to the invention.

In addition, the invention provides a pharmaceutical composition comprising a binding moiety, multivalent reagent or multispecific reagent according to the invention and a pharmaceutically acceptable carrier, diluent, adjuvant and/or immunostimulant.

The invention also provides a method of treating a pathological condition in a subject, which method comprises administering to the subject binding moiety, multivalent reagent or multispecific reagent according to the invention.

The invention also provides a method of selecting a binding moiety with an affinity for a target molecule which comprises
(i) providing a plurality of polynucleotides encoding binding moieties comprising a CBD, which polynucleotides comprise one or more modifications in the CBD;
(ii) expressing the binding moieties encoded by the polynucleotides; and
(iii) selecting one or more binding moieties having an affinity for the target molecule.

The invention also provides a polynucleotide library comprising a plurality of polynucleotides encoding binding moieties comprising a cytokine binding domain, which polynucleotides comprise one or more modifications in the cytokine biding domain.

The invention also provides expression vectors useful in the generation of binding moieties according to the invention. Accordingly, the invention provides an expression vector comprising:
a) a first nucleic acid sequence encoding a CBD;
b) an insertion site in a region between the ends of the first nucleic acid sequence, the insertion site comprising a nucleotide sequence unique to said expression vector which is cleaved by a restriction endonuclease and which allows a second nucleic acid sequence encoding an amino acid sequence to be inserted into the first nucleic acid to encode a modified CBD; and
c) a regulatory control sequence operably linked to said first nucleic acid sequence which directs expression of the first nucleic acid sequence.
   Preferably, the region encodes a solvent exposed region, preferably a loop.
   The invention also provides an expression vector comprising:
a) a first nucleic acid sequence encoding a CBD, said sequence comprising a deletion in a region between the ends of the first nucleic acid sequence;
b) an insertion site in place of the deleted sequence which site allows a second nucleic acid sequence encoding an amino acid sequence to be inserted into the first nucleic acid to encode a modified CBD.
c) a regulatory control sequence operably linked to said first nucleic acid sequence which directs expression of the first nucleic acid sequence.
   Preferably, the region encodes a solvent exposed region, preferably a loop.
   The invention also provides an expression vector comprising:
a) a first nucleic acid sequence encoding a CBD;
b) a number of insertion sites in regions between the ends of the first nucleic acid sequence, each insertion site comprising a nucleotide sequence unique to said expression vector which is cleaved by a restriction endonuclease and which allows a nucleic acid sequence encoding an amino acid sequence to be inserted into the first nucleic acid to encode a modified CBD.
   Preferably, one or more regions, preferably each region, encodes a solvent exposed region, preferably a loop.

The invention also provides a nucleic acid sequence encoding a peptide display scaffold comprising:
a) a first scaffold sequence encoding a CBD; and
b) a second sequence encoding a peptide and inserted at a site located in a region of said first scaffold sequence encoding a cytokine binding loop.

The invention also provides an expression vector comprising a nucleic acid sequence according to the invention described immediately above, as well as a CDB display library comprising a plurality of said expression vectors.

The invention also provides a polypeptide encoded by the nucleic acid sequence according to the invention described above, as well as a protein multimer comprising at least two of said polypeptides.

The invention also provides a method of identifying a modified CBD which binds to a target molecule of interest, which method comprises:
(i) providing a CBD display library of the invention;
(ii) expressing the polypeptides encoded by the polynucleotides; and
(iii) selecting one or more polypeptides that bind to the target molecule.

The various features and embodiments of the present invention, referred to in individual sections above apply, as appropriate, to other sections, mutatis mutandis. Consequently features specified in one section may be combined with features specified in other sections, as appropriate.

### DESCRIPTION OF THE FIGURES

FIG. 1. (a) A coil representation of the backbone of a CBD, illustrated by the CBD of the IL-6 receptor (IL-6R) (D2 representing the N-terminal domain and D3 representing the C-terminal domain). The loops marked L1 to L4 and L5 to L7 respectively, represent the loops from the N-terminal and C-terminal domains of the CBD that can engage a target macromolecule: (b) The view with the molecule rotated 90° with the seven loops facing up: (c) A coil representation of the backbone of the variable domains of the heavy and light chains of the Fv domain of an immunoglobulin, illustrated by the NC10 anti-neuraminidase Fv domain, showing the Fv domain's respective antigen binding CDRs as loops L1, L2, L3, H1, H2 and. (d) the view with the molecule rotated 90° with the loops facing up.

Both molecules are draw to scale, and it can been seen that while the Fv antigen binding site is approximately isotropic in distribution, the CBD loops are long and narrow, offering a different type of surface topology when compared to the potential binding site of antibody molecules.

FIG. 1A: A schematic representation of a binding moiety according to the present invention. The CBD-like scaffold structure consists of a first and a second FnIII-like domain (indicated as FnIII¹ and FnIII²). Solvent exposed loops present on each FnIII-like domains define a binding region capable of association with a target molecule. The binding region is essentially defined by solvent exposed loops presented by both domains.

FIG. 2. (a) A ribbon diagram of the CBD of IL-6R, showing the β-sheet arrangement of the two FnIII domains, and the cytokine binding loops L1 to L7. (b) the same as in (a) but rotated 90° with the loops facing up.

FIG. 3. (a) The amino acid sequence of IL-6R extracellular domain, showing the CBD comprising domain D2 (residues 92 to 195) and domain D3 (residues 196 to 297). The position of β-sheet structures are indicated by #. The position of loops in the cytokine binding region are shown by * and marked L1 to L7. The Pro94, Pro95, Cys102, Cys103, Trp115, Cys146, Cys157, Pro199, Pro200, Trp219, Arg274, Trp284, Ser285, Trp287 and Ser288 residues are all conserved in known CBDs. The Leu100, Leu108, Val111, Ala127, Leu129, Val131, Leu159, Tyr169, Val171, Met173, Val175, Phe189, Gly191, Ile194, Leu195, Pro197, Ile203, Val205, Leu215, Val217, Leu32, Phe234, Leu236, Tyr238, Phe246, Trp249, Ile260, Ala263, Val271, Leu273, and Glu286 residues are mainly conserved hydrophobic residues in known CBDs. The Pro98, Pro117, Trp225, Cys258, His269, Ala291 and Gly293 are, in the majority, conserved residues in all known CBDs.

FIG. 3(b) depicts the sequence alignment of the CBDs from IL-6R, IL-11R, PRLR and GCSR. Loops L1 to L7 are outlined by boxes.

FIG. 4. The CBD of IL-6R with domain D3 (lower part—shade 1) and domain D2 (top part—shade 2), with the loop residues from D3 (shade 3) and from D2 (shade 4). Shades 1 to 4 are of increasing darkness. (a) and (c) have CPK and loop representations of the cytokine binding region loops L1 to L7. (b) and (d) are the same as in (a) and (c) but rotated 90° with the loops facing up.

FIG. 5. Comparison of the sequences of CBDs from 77 known genes. FIG. 5A compares the sequences in the “first” FnIII domain, containing loops 1 to 4, and FIG. 5B the sequences in the “second” FnIII domain, containing the loops 5-7. Conserved residues as described in Example 3 for the IL-6 receptor are aligned according to their sequence homologies. For example the hydrophobic residues, the cysteine residues (C) and in some cases two prolines side by side (PP) are aligned. The location of the 7 binding loops is indicated by the double-headed arrows.

FIG. 6. The backbone of the CBD of IL-6R, with the cytokine binding loops L1 to L7 coloured dark. In (a) and (b) a CPK representation the residues that are conserved in all known CBDs. In (c) and (d) including a CPK representation of all residues which are almost always conserved and mainly hydrophobic.

FIG. 7. Pictorial representation of the scaffold, firstly demonstrating the structural similarities of the IL-6R, prolactin receptor and the novel scaffold, and secondly the close structural alignment of all three as shown in the central picture.

### DETAILED DESCRIPTION OF THE INVENTION

Unless defined otherwise, all technical and scientific terms used herein have the same meaning as commonly understood by one of ordinary skill in the art (e.g., in molecular biology and biochemistry). Standard techniques are used for molecular and biochemical methods (see generally, Sambrook et al., Molecular Cloning: A Laboratory Manual, 3rd ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

Throughout the specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

By “hydrophobic residues” or “nonpolar residues” as used herein is meant valine, leucine, isoleucine, methionine, phenylalanine, tyrosine, and tryptophan.

By “polar residues” herein is meant serine, threonine, histidine, aspartic acid, asparagine, glutamic acid, glutamine, arginine, and lysine.

By “extracellular domain” of as used herein is meant a segment of a protein existing predominantly outside the cell. For transmembrane proteins, this segment can be tethered to the cell through a transmembrane domain or released from the cell through proteolytic digestion. Alternatively, the extracellular domain could comprise the whole protein or amino acid segments thereof when secreted from the cell.

### Cytokine Binding Domains (CBDs)

A cytokine binding domain is defined herein as a polypeptide consisting of a first and a second FnIII-like domain. The FnIII-like domains are each independently domains having immunoglobulin folds in a FnIII-like association of beta sheets. The two domains lie on a similar plane and are typically connected at about 90° to each other. Preferably, at least one domain comprises a tryptophan-arginine ladder region, which preferably comprises a Trp-Ser-X-Trp-Ser (“WSXWS”) motif or variant thereof which forms a left-handed 3₁₀ helix.

Each FnIII-like domain comprises a number of loops, typically surface and solvent exposed loops. The loops in the two domains making up the CBD are arranged in a substantially linear manner over the two domains to form, and to substantially define, a binding region.

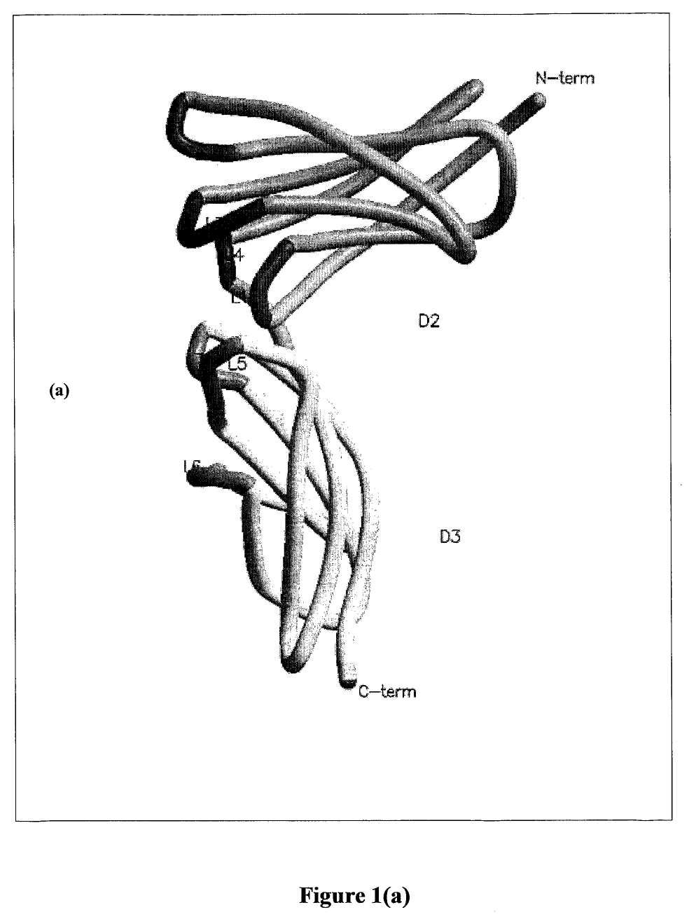

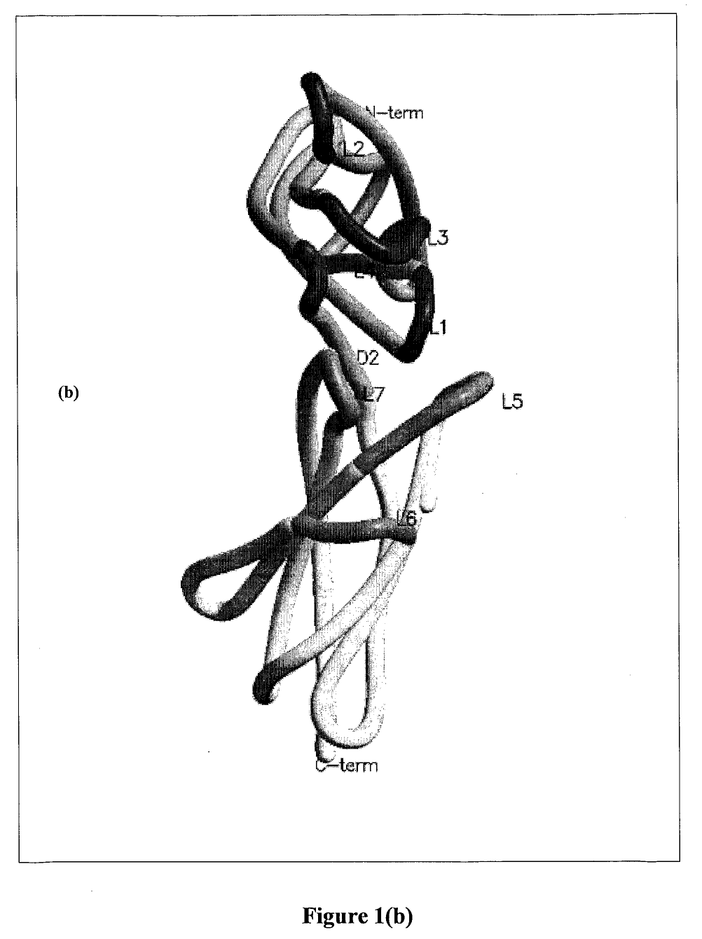

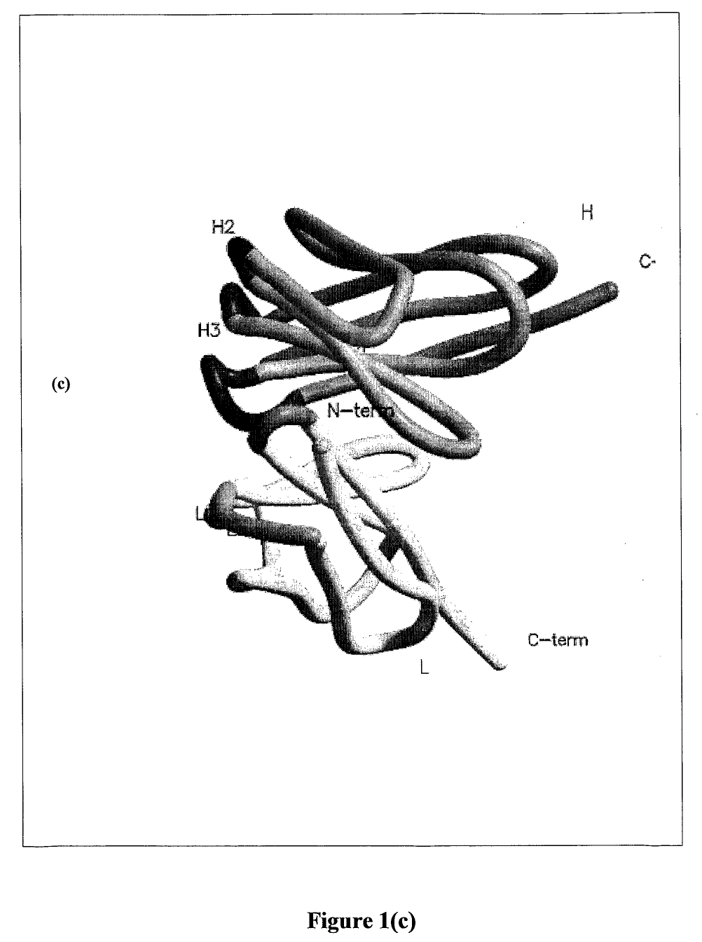

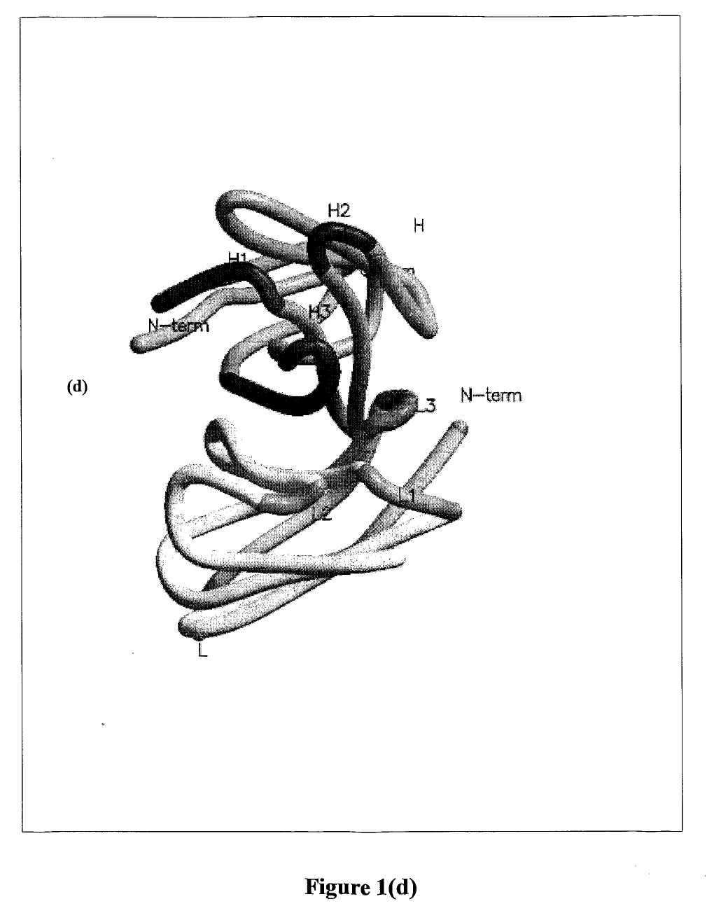

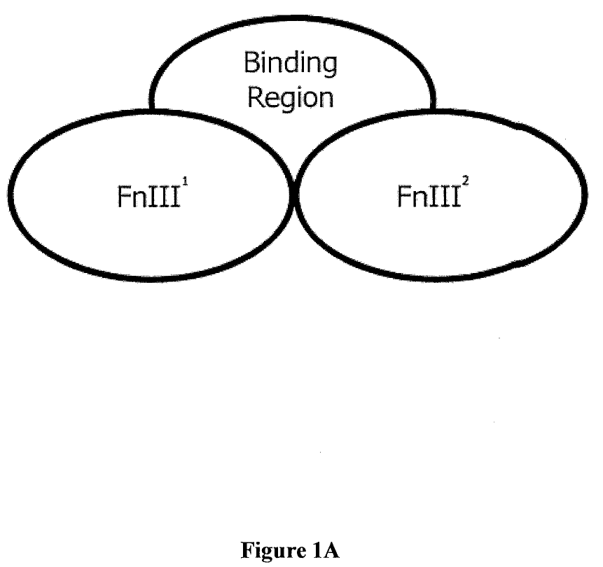

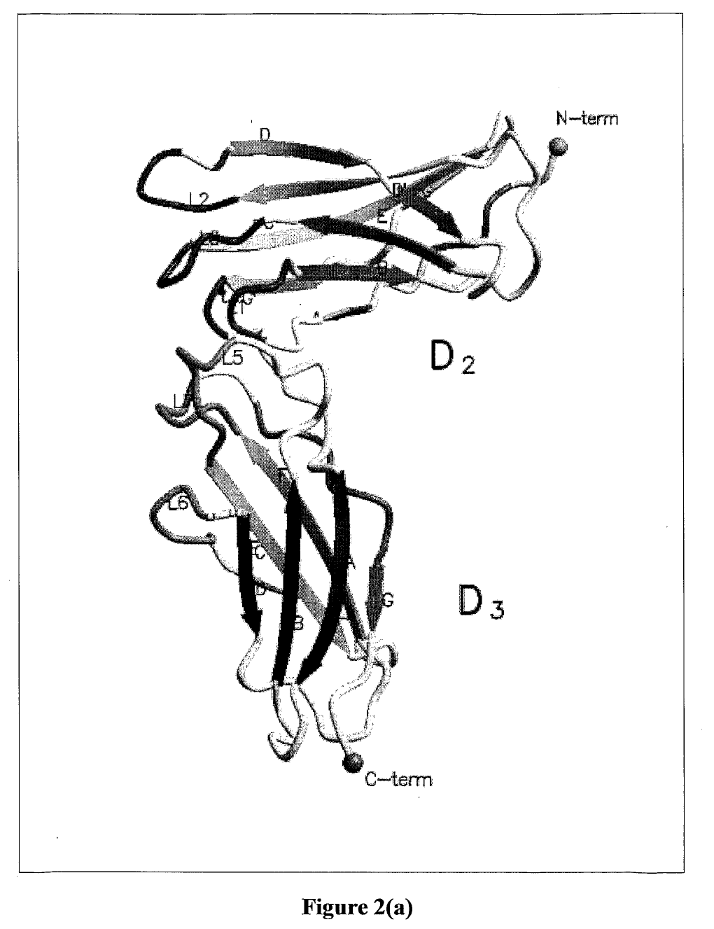

The structural definition of CBDs given above is further illustrated and supported by reference to FIGS. 1-6. In particular, it is further illustrated and supported by reference to the primary, secondary and tertiary structure, including the three dimensional structure, of IL-6R as presented in FIGS. 1-6 and detailed in Varghese J N et al., 2002, PNAS 99(25):15959-15964 and PCT/AU02/01255, the entire contents of which are herein incorporated by reference. These references also provide the atomic coordinates of the extracellular domain of IL-6R. FIGS. 1-6 and the aforementioned references variously provide details of structural features, including the arrangement of beta sheets, the orientation of each of the two domains with respect to one another and the location of the solvent exposed loops that are typically present in CDBs.

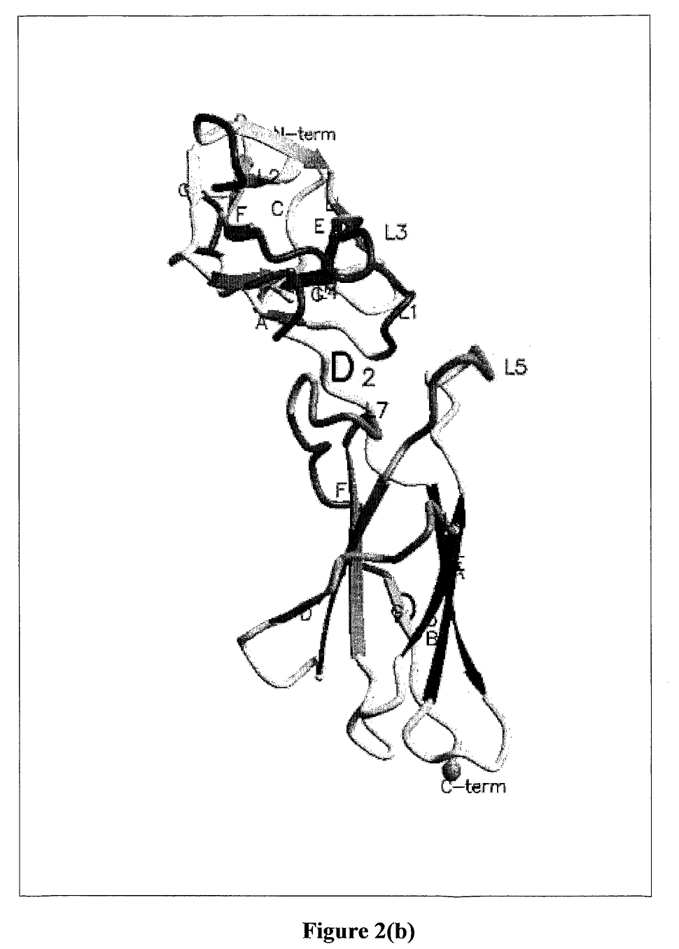

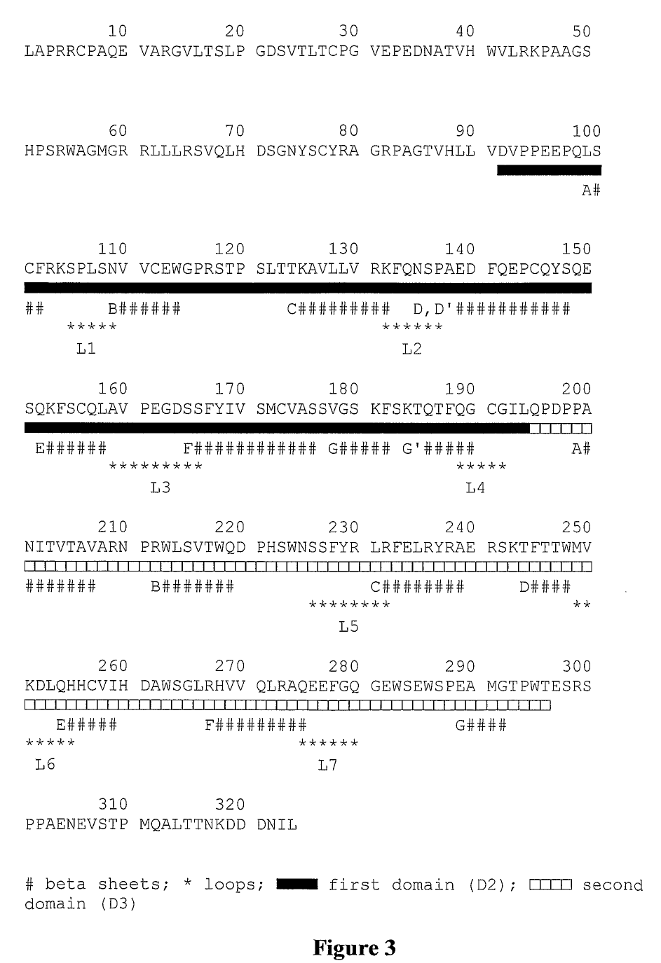

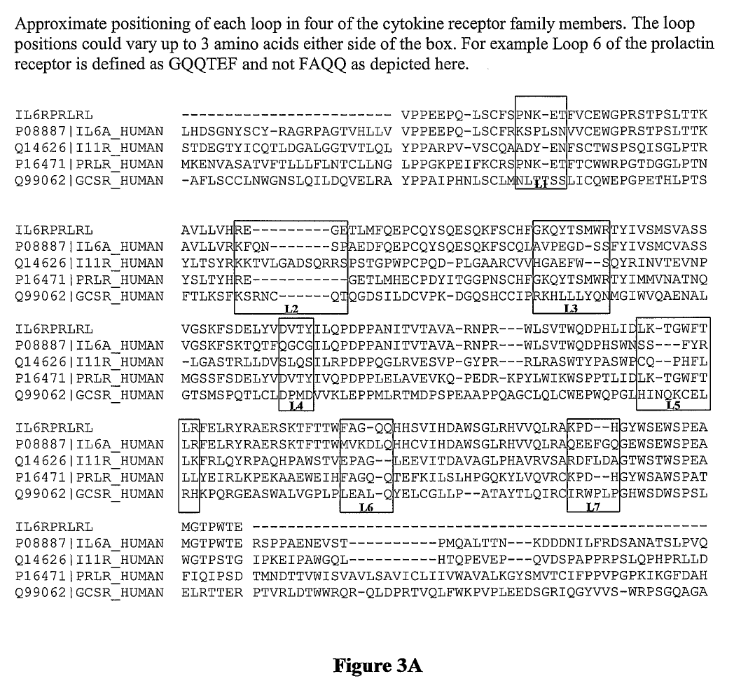

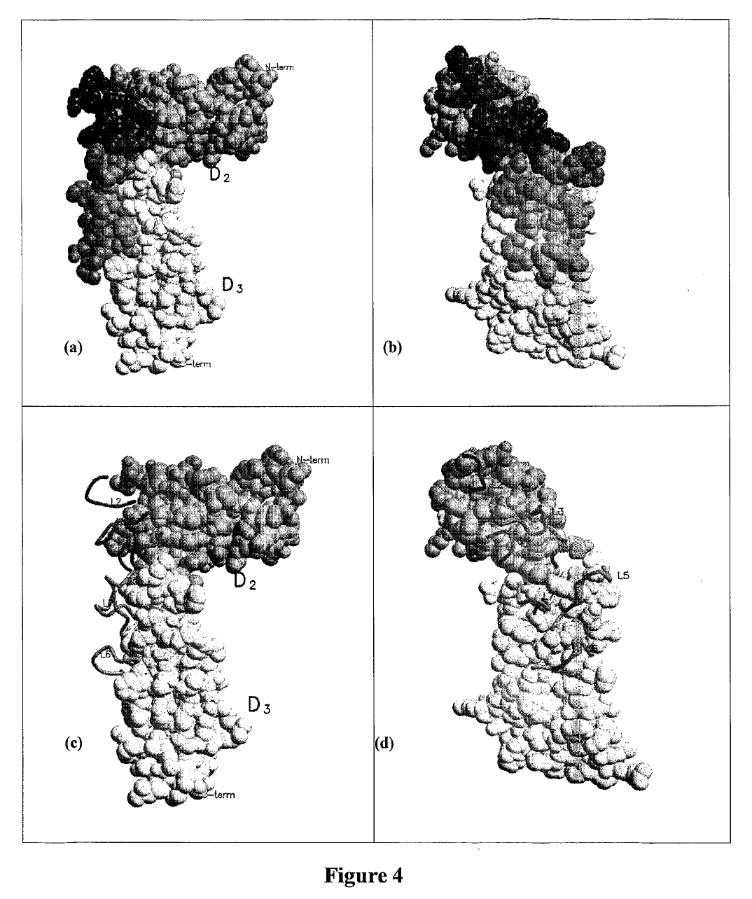

The amino acid sequence of IL-6R is given in FIG. 3, which also highlights the location of various secondary structures in the primary sequence. The CBD of IL-6R is defined by the D2 and D3 domains (amino acids 92 to 297). The two domains lie on a similar plane to form a long flat structure in which the D2 and D3 domains are connected at about 90° to each other. The D2 domain comprises 4 solvent exposed loops (L1: Lys105 to Asn110; L2: Lys133 to Glu140; L3: Ala160 to Phe168; and L4: Gln190 to Gly193) and the D3 domain comprises 3 solvent exposed loops (L5: Asn226 to Arg233; L6: Met250 to His256; and L7: Gln276 to Gln281), which together form a long and narrow binding area held in place by the rigid D2 and D3 framework of the CBD. The location of these loops in the three-dimensional structure of folded IL-6R is shown in FIG. 4.

Arg239, Phe246, Arg237, Trp287, Arg274, Trp284 and Gln276 together form the tryptophan-arginine ladder region, which comprises a WSXWS motif.

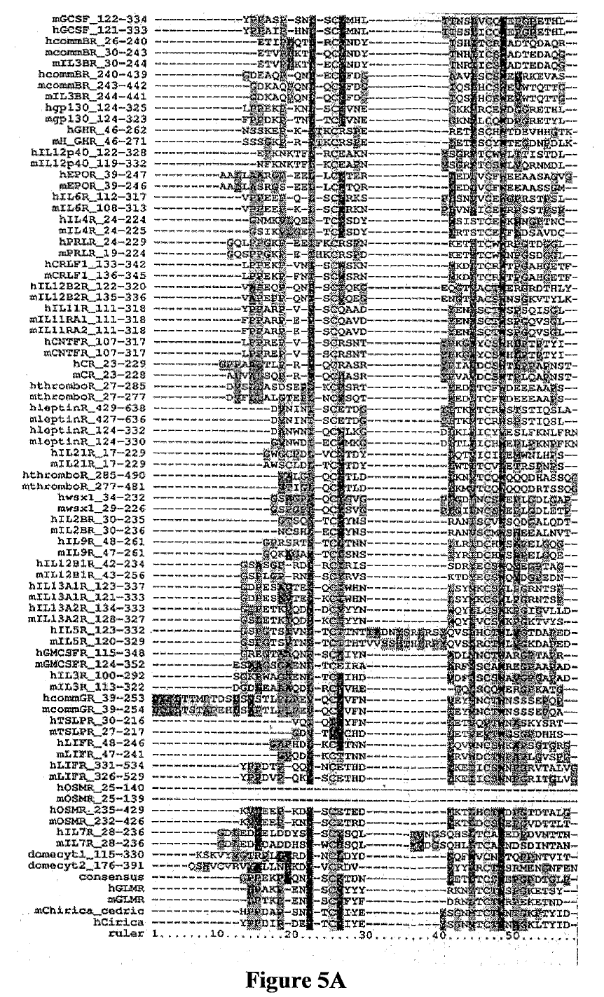

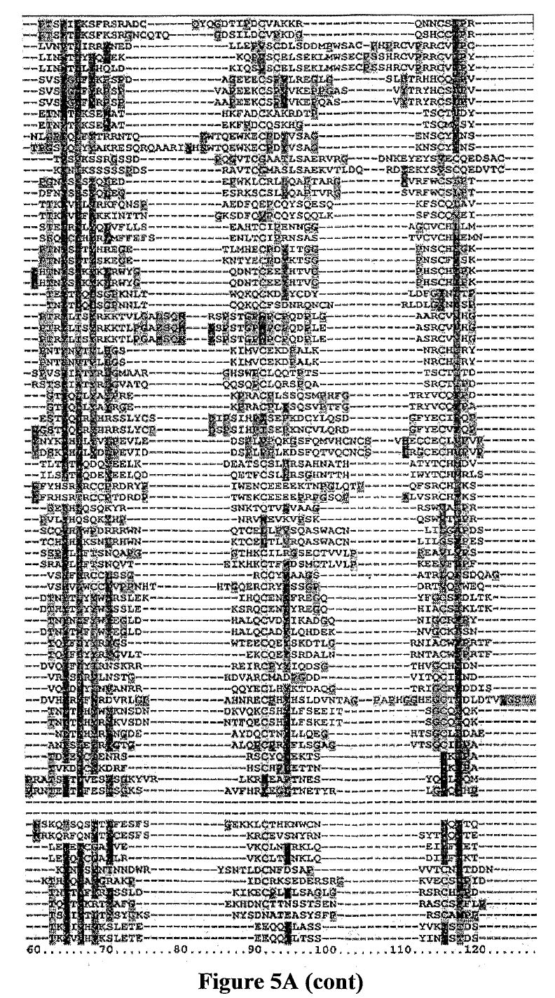

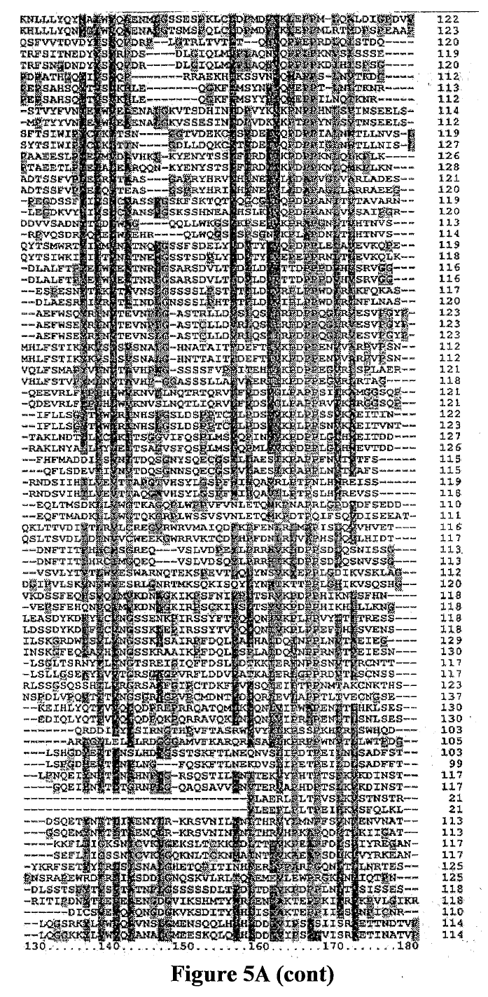

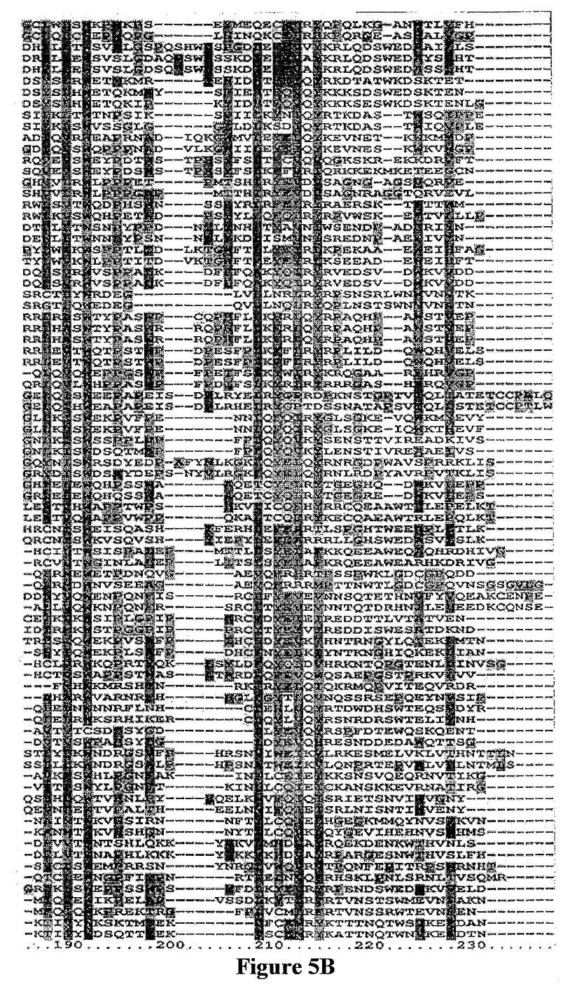

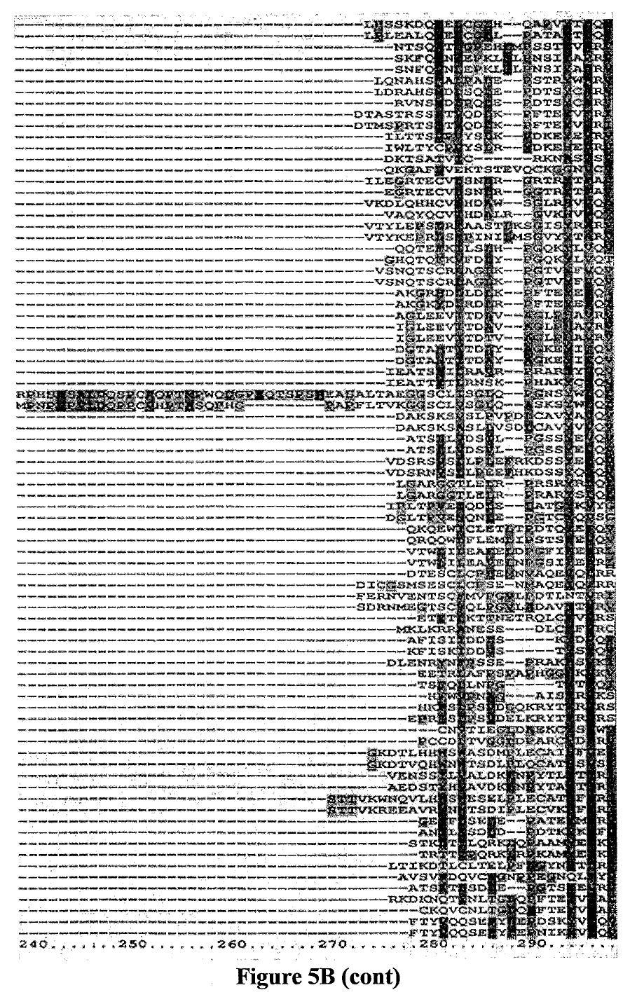

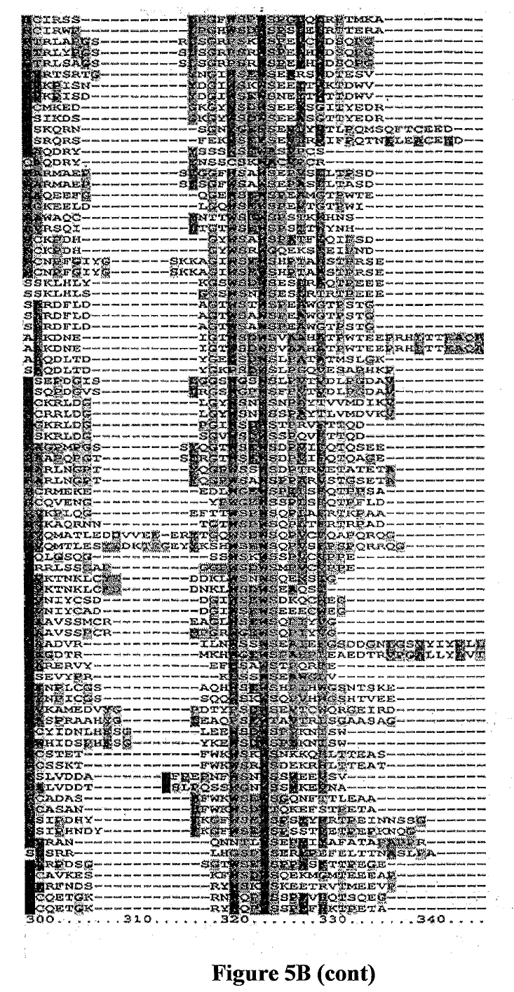

The alignment of CBDs present in over seventy gene products is shown in FIG. 5. FIG. 5A depicts the sequence alignment of the first FnIII-like domain (corresponding to D2 of the IL-6R CBD), defined over location R1 to approximately R180 as numbered in FIG. 5. FIG. 5B depicts the sequence alignment of the second FnIII-like domain (corresponding to D3 of the IL-6R CBD), defined over location approximately R185 to R99 as numbered in FIG. 5. The hinge connecting the first and second FnIII-like domains is defined over the approximate location of R180 to R185, e.g. from R181 to R184, as numbered in FIG. 5. The hinge region typically comprises residues flanking the side of loop L4.

The alignments in FIGS. 5A and 5B clearly demonstrate a high degree of conservation. For example, cysteine residues, hydrophobic amino acid residues, hydroxylated amino acid residues, proline/glycine residues, acidic amino acid residues and basic amino acid residues are all variously conserved. Examples of conserved amino acid residues found in the alignments of FIG. 5 are given in Table 1.

TABLE 1
Examples of conserved amino acid residues
found in the alignments of FIG. 5.

| Conserved | | FnIII-like |
|---|---|---|
| residues | Location in FIG. 5 | domain |
| Cys | R25, R46, R91, R115 | First |
| Hydrophobic | R22, R26, R41, R44, R48, R64, | First |
| | R66, R117, R136, R138, R140, | |
| | R142, R146, R156, R158, R161, | |
| | R162, R170, R172 | |
| | R187, R189, R191, R197, R208, | Second |
| | R210, R212, R214, R224, R227, | |
| | R280, R282, R285, R287, R295, | |
| | R297, R299, R319, R322, R326, | |
| | R328 | |
| Hydroxylated | R47, R62, R64, R68, R70, R94, | First |
| (Tyr, Thr, Ser | R136 | |
| and including His) | R210, R214, R203, R320, R323, | Second |
| | R330 | |
| Pro/gly | R14, R15, R18, R50, R51, R164, | First |
| | R166, R167 | |
| | R185, R193, R195, R198, R199, | Second |
| | R216, R218, R177, R289, R290, | |
| | R317, R324, R325 | |
| Acidic | R211, R321 | Second |
| Basic | R298 | Second |

Table 1 is not intended to be a comprehensive analysis of the degree of conversation across the CBD sequences shown in FIG. 5. It merely indicates some of the positions where conservation is occurring and serves to demonstrate the extent of conservation. The skilled person will appreciate that there are other positions and complexities of conservation present in the aligned sequences in FIG. 5 and will be able to elucidate these using knowledge and analytical tools that are routinely available to them.

FIG. 5 also demonstrates that certain motifs, such as the WSXWS motif, are present in the vast majority of CDBs (see, for example, location R321-R325). Particularly significantly, all the sequences have 7 loops corresponding to loops L1 to L7 identified and discussed above in relation to IL-6R above. Table 2 details the approximate locations of these loops as found in FIG. 5. It will be understood that loops may also comprise one or more amino acids flanking the locations in FIG. 5 as defined in Table 2. Suitably, the loops may comprise up to 10, preferably up to 5 and more preferably up to 4 flanking amino acids

TABLE 2
Location of loops L1 to L7 in FIG. 5.

| Loop | Location in FIG. 5 | FnIII-like domain |
|---|---|---|
| L1 | R28-43 | First |
| L2 | R70-87 | First |
| L3 | R118-135 | First |
| L4 | R157-160 | First |
| L5 | R198-209 | Second |
| L6 | R228-278 | Second |
| L7 | R300-316 | Second |

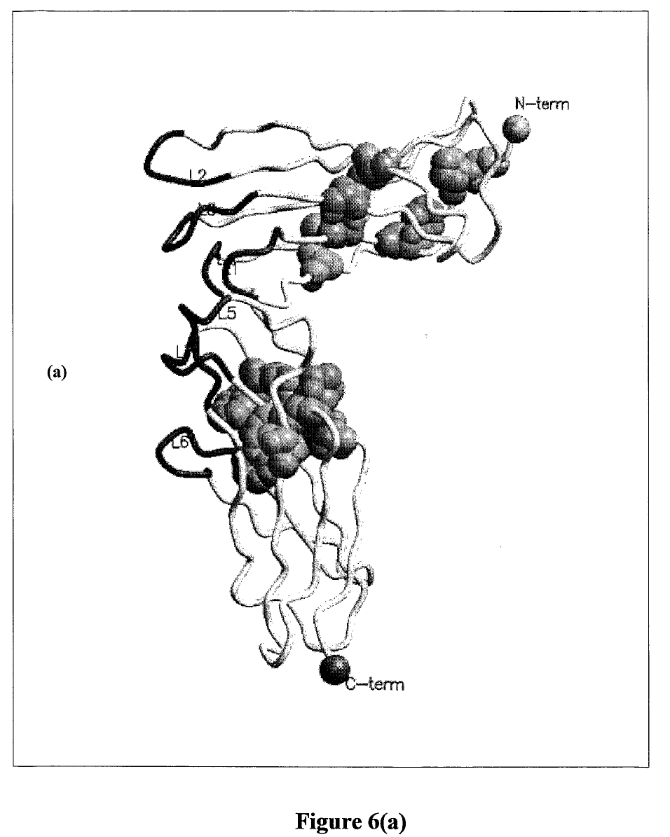

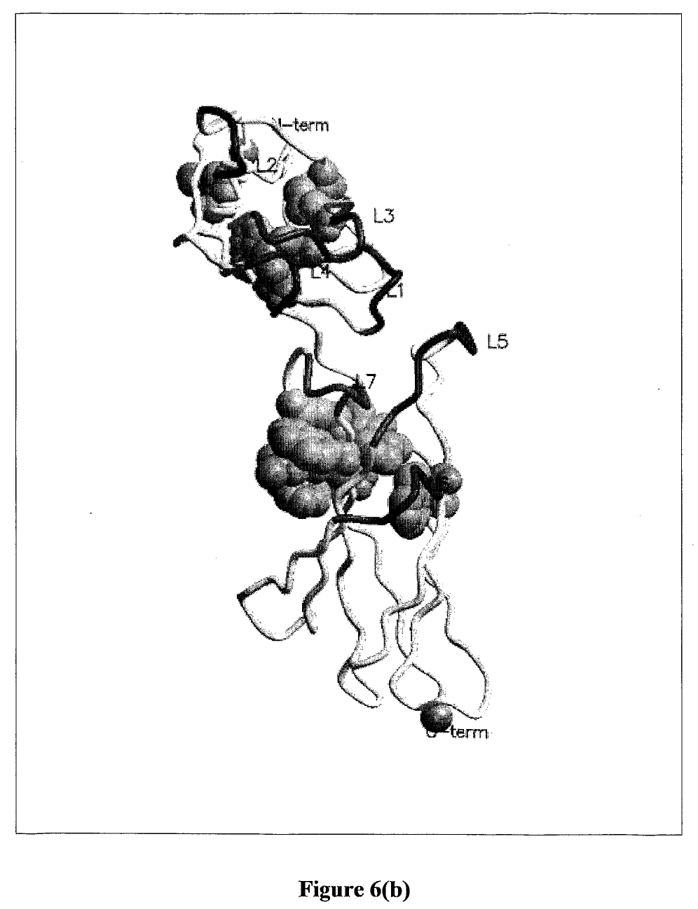

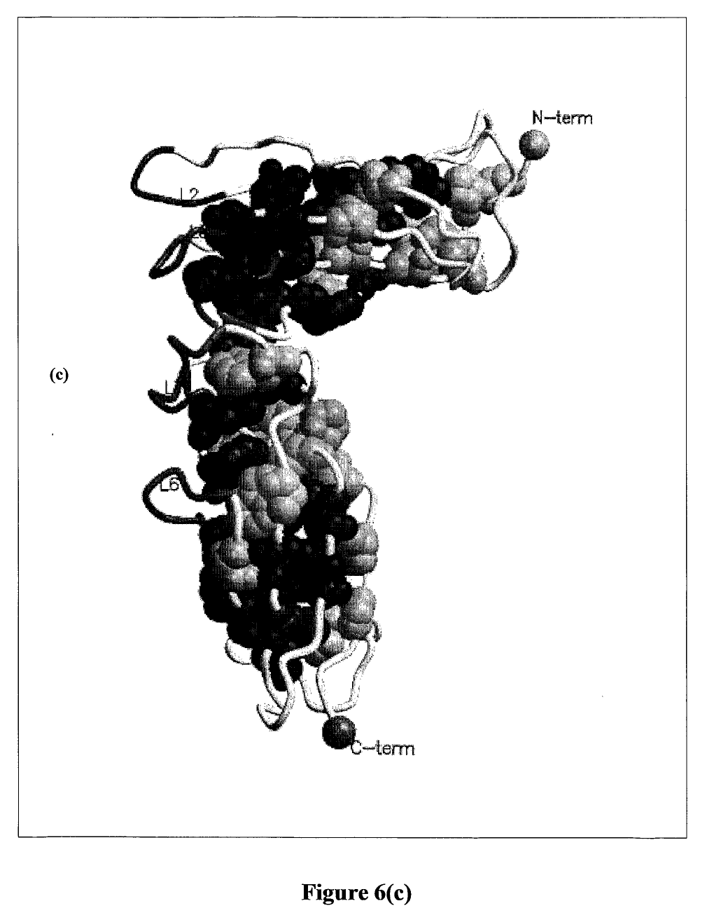

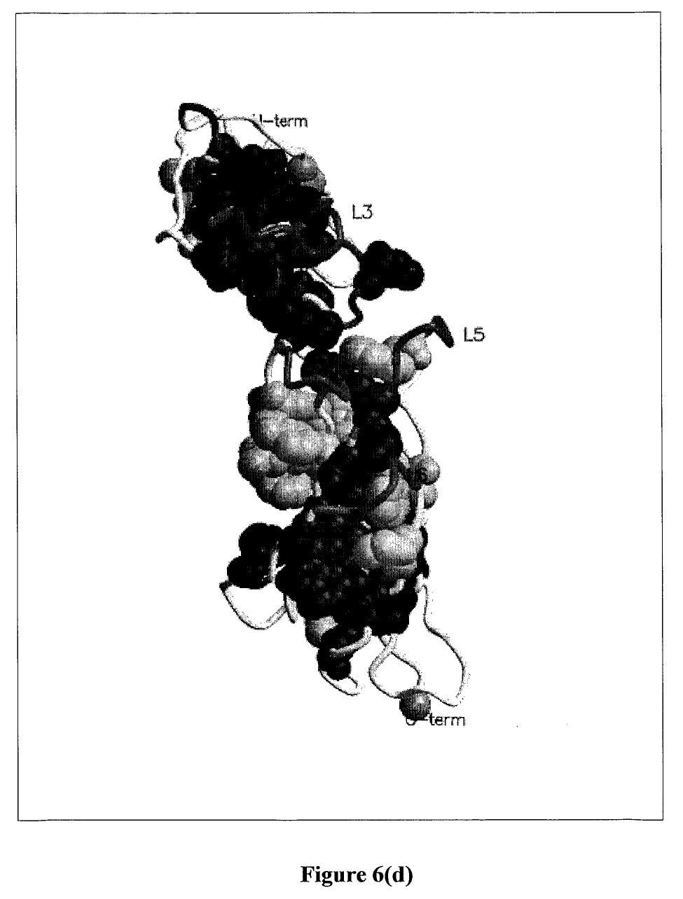

It will be understood that FnIII-like domains may be derived from proteins not specifically disclosed herein. Furthermore, the skilled person will have no difficulties identifying such other suitable FnIII-like domains within CBDs from other proteins. A number of methods have been described for identifying protein sequences of suitable structure and function. These methods include, but are not limited to, sequence alignment methods, structure alignment methods, sequence profiling methods and energy calculation methods. It is evident from the alignments presented in FIG. 5 and from structural information and published crystallographical data (for example Aritomi M. et al., Nature, 1999, 401(6754):713-7; Bravo J. et al., EMBO J., 1998, 17(6):1665-74; Elkins P. A. et al., Cell, 1999, 97(2):271-81; Josephson K. et al., Immunity, 2001, 15(1):35-46; Man D. et al., J. Biol. Chem., 2003, 278(26):23285-94; Schreuder H. et al., Nature, 1997, 386(6621):194-200) that the CBD structure exemplified by IL-6R is conserved in other CBDs. Thus, CBDs can be defined with reference to the three-dimensional structure of domains D2 and D3 of IL-6R, in particular with reference to the structural coordinates of the backbone carbon atoms of IL-6R as provided in Varghese J N et al., 2002 PNAS 99(25):15959-15964 and PCT/AU02/01255. Thus, as new crystal structures are solved, it will become immediately apparent if a protein contains a CBD comprising FnIII-like domains by comparing sequence and structural (secondary and tertiary) data with, for example, that of IL-6R and other proteins listed in FIG. 5. However, it will be appreciated that the three-dimensional structure of other CBDs will not correspond precisely to that of the IL-6R. FIG. 6 illustrates in the context of the IL-6R, the regions of the CBD structure that are most highly conserved in known naturally occurring CBDs.

Alternatively and/or additionally, suitable CBDs may be identified through sequence alignment analysis with the sequences in FIG. 5. It will be readily apparent to the skilled person upon carrying out a suitable alignment analysis whether the protein comprises a CBD having two FnIII-like domains. The amino acid sequence of a potential CBD can be directly compared with the sequences in FIG. 5 and in particular those residues known to be highly conserved for known CBDs as described above. After aligning the conserved residues, allowing for necessary insertions and deletions in order to maintain alignment (i.e. avoiding the elimination of conserved residues through arbitrary deletion and insertion), any residues equivalent to particular conserved amino acids in the sequences of FIG. 5 should become defined. Furthermore, any sequence motifs should also be identified as should regions where loop structures are likely to occur (i.e. regions where there is little or no predicted secondary structure and which are relatively polar in nature).

Suitable computational methods for carrying out such analyses to identify protein sequences having the desired structural and functional properties are well known in the art and include, for example, Modeller.

Preferably, the first FnIII-like domain of the CBD comprises four loops located at positions L1 to L4 as indicated in FIG. 5A when the amino acid sequence is aligned with the sequences in FIG. 5.

Preferably, the second FnIII-like domain of the CBDs of the present invention comprises three loops located at positions L5 to L7 as indicated in FIG. 5B when the amino acid sequence is aligned with the sequences in FIG. 5.

Preferably, the second FnIII-like domain comprises a tryptophan-arginine ladder region, which preferably comprises a WSXWS motif or variant thereof.

Preferably, the first FnIII-like domain comprises four loops located at positions L1 to L4 as indicated in FIG. 5A and the second FnIII-like domain comprises three loops located at positions L5 to L7 as indicated in FIG. 5B when the amino acid sequence is aligned with the sequences in FIG. 5.

The presence of loops L1 to L4 and L5 to L7, and, if present, a tryptophan-arginine ladder would be evident from a suitably performed sequence alignment and analysis.

As an alternative to FIG. 5, it is also possible to identify suitable CBDs through homology of the primary sequence with FIG. 3 in the same way as described above in relation to FIG. 5.

Where crystal structure data is not available, computer modelling tools are now routinely available that allow potentially useful CBD candidates to be modelled and their predicted structures to be directly compared with, for example, the CBD of IL-6R. Therefore, in addition to being able to identify whether a protein contains two FnIII-like domains presenting the loops identified in FIG. 5 at analogous positions along the primary sequence and preferably possessing other motifs such as a tryptophan-arginine ladder region, which preferably comprises a WSXWS motif or variant thereof, it is also possible for the tertiary structure of the protein, or at least the relevant domain of the protein, to be computer modelled and that 3-D model compared with known crystal structures of CBDs, such as the IL-6R CBD. In this way, the spatial correlation of the loops in the protein of interest can be compared with that in known CBDs.

Although FIGS. 1, 2, 3, 5 and 6 mention seven loops, it will be understood that the loop given as L4 (corresponding to A190 to G193 of IL-6R and located at R154-R160 in FIG. 5) is small and in some literature may not always be referred to as a loop per se. It has been included in the present description for the sake of completeness. However, this does not mean that the present invention excludes CBDs described in the literature as comprising six loops. On the contrary, such CBDs may evidently be within the scope of the present invention.

The FnIII-like domains of the CBDs of the binding moieties may be derived from any suitable naturally occurring CBDs. Examples of suitable naturally occurring CBDs are listed in FIG. 5. Preferably, the CBDs are derived from the extracellular domains of growth factor and cytokine receptor family members, and in particular cytokine receptor family members and associated proteins such as, for example, gp130. Preferred cytokine receptor family members are those in class I (hematopoietin receptors) or class II, preferably class I. Examples of suitable proteins from which CBDs may be derived include the IL-Rs (interleukin receptors), G-CSFR (granulocyte colony stimulating factor receptor), GM-CSFR (granulocyte macrophage colony stimulating factor receptor), PRLR (prolactin receptor), LIFR (leukemia inhibitory factor receptor), OSMR (oncostatin M receptor), cardiotrophin CT-1 receptor, CNTFR (ciliary neutrotrophic factor receptor), leptin receptor, EPOR (erythropoietin receptor), gp130, GHR (growth hormone receptor) and stromal lymphopoietin protein receptor. The numbering of the amino acid residues that constitute the CBD for many of these proteins is provided in FIG. 5.

Examples of suitable IL (interleukin) receptors include the IL-2R, IL-3R, IL-4R, IL-5R, IL-6R, IL-7R, IL-9R, IL-11R, IL-12R, IL-13R, IL-15R and IL-21R.

For the avoidance of doubt, with regards to cytokine receptors having alpha and beta subunits, any extracellular domains referred to herein from which suitable CBDs may be derived are alpha subunit extracellular domains, not beta subunit domains.

The FnIII-like domains of a CBD of the invention can be derived from the same or different sources. For example, the first FnIII-like domain may be derived from one protein and the second FnIII-like domain derived from a different protein. For example, the first domain of IL-11R could be combined with the second domain of IL-12R. Similar pairing could also be performed with IL-5R and IL-4R and with prolactin and GMCSFR. Where the two FnIII-like domains are derived from different proteins, it will be appreciated by the skilled person that they must be suitably orientated with respect to each. The first FnIII-like domain should be suitably hinged to the second FnIII-like domain so that the domains lie in a similar plane, the domains being orientated with respect to each other as they would be to their respective other FnIII-like domain in the native protein from which they derive.

Linkers used to link protein domains are well-know and well understood in the art, in particular in relation to proteins in the immunoglobulin superfamiles. Therefore, the skilled person will appreciate that any suitable hinge may be used to connect the two FnIII-like domains. The two FnIII-like domains can be linked by genetic or chemical means. Examples of suitable chemical linkage include linking the two domains using a suitable cross-linker such as dimaleimide. Alternatively, the two domains may be linked by providing cysteine residues at the respective C- and N-terminals and forming a disulphide bond. In addition, they could be linked using single chain GlySer linkers such as GlyGlyGlyGlySer. The domains may also be linked genetically. For example, where a restriction enzyme (RE) site naturally occurs between loops 4 and 5 in a wild type CBD, this site can be used to link the two domains. Alternatively, a suitable RE site may be introduced between loops 4 and 5. Preferably, any RE site will lie between that part of the sequence encoding the region of the FnIII-like domains between the end of the beta sheet immediately following loop 4 and the beginning of any beta sheet immediately preceding loop 5.

FIG. 5 presents numerous examples of naturally occurring hinges in CBDs. Preferably, the hinge is a stretch of from about 3 to 15 amino acids, preferably from about 4 to 10 amino acids, situated between the two FnIII-like domains. The hinge connects loop 4 to loop 5 via the respective N- and C-terminals of the two domains. Preferably, the hinge is derived from one of the sources from which one of the FnIII-like domains is derived.

It will be apparent that the binding moieties of the invention can be generated de novo based on the structural constraints for a CBD described here and above.

In a preferred embodiment, the two FnIII-like domains of a CBD are derived from the same source protein.

### Binding Moieties

The binding moieties of the present invention comprise an extracellular CBD consisting of a first FnIII-like domain and a second FnIII-like domain in which the CBD comprises a modification which alters at least one property of the CBD. It will be understood that the binding moieties of the present invention do not encompass and do not relate to the full-length, wild-type proteins from which suitable FnIII-like domains may be derived. Rather, they encompass and relate to portions of CBD-containing receptors, preferably the extracellular portions, which have been removed or isolated from their natural environments. Where the binding moieties are derived from the extracellular portion of a CBD-containing receptor, the binding moieties are preferably no larger in terms of the number of amino acid residues and/or molecular weight than the native extracellular domain from which the FnIII-like domain(s) is/are derived.

In a preferred embodiment, the CBD of the binding moiety accounts for at least 50%, preferably at least 60%, more preferably at least 70%, yet more preferably at least 80%, even more preferably at least 90% and most preferably at least 95% of the total molecular weight of and/or number of amino acid residues in the binding moiety. In a particularly preferred embodiment, the binding moiety consists essentially of the CBD.

Preferably, the only binding domains present in the binding moieties of the present invention are the two FnIII-like domains. The two FnIII-like binding domains form a single binding region. The binding moieties of the present invention are therefore monomeric polypeptide or protein bodies.

### Altered Properties

The CBD is modified such that a property of the CBD is altered.

A property of a cytokine binding domain is altered if any characteristic or attribute of the cytokine binding domain differs from the corresponding property of the unmodified cytokine binding domain. These properties include, but are not limited to, substrate specificity, substrate affinity, binding affinity, binding selectivity, catalytic activity, thermal stability, alkaline stability, pH activity profile, resistance to proteolytic degradation, kinetic association, kinetic dissociation, immunogenicity, ability to be secreted, ability to activate receptors, ability to treat disease, solubility, cytotoxic activity and oxidative stability.

Unless otherwise specified, a property of a cytokine binding domain is considered to be altered when the property exhibits at least a 5%, preferably at least 10%, more preferably at least a 20%, yet more preferably at least a 50%, and most preferably at least a 2-fold increase or decrease relative to the corresponding property in the unmodified cytokine binding domain.

In a preferred embodiment, the solubility of the modified CBD, and concomitantly the binding moiety, is altered, preferably improved, relative to the corresponding unmodified CBD (i.e. the unmodified binding moiety).

In another preferred embodiment, the stability of the CBD is altered, preferably improved, relative to the corresponding unmodified CBD. Examples of altering the stability include changing one of the following properties:—thermal stability, alkaline stability, pH activity profile and resistance to proteolytic degradation.

In a particularly preferred embodiment, the binding characteristics of the CDB are altered. Examples of altering the binding characteristics include changing one of the following properties: substrate specificity, substrate affinity, catalytic activity, kinetic association, kinetic dissociation, binding affinity and binding selectivity.

### Modifications

By modifying the cytokine binding domain we mean introducing at least one modification into a wild type FnIII domain from a wild type cytokine binding domain sequence.

By “wild-type cytokine binding domain” we mean a cytokine binding domain that is found in nature and includes allelic variations; that is, an amino acid sequence that has not been intentionally modified. The wild type cytokine binding domain sequence may be derived from any species, preferably a mammalian species. In a preferred embodiment, the wild-type cytokine binding domain has a sequence as shown in FIG. 5.

Suitable modifications include substitutions, insertions and deletions within at least one specified region.

Preferably, the size and/or area of the CBD is altered as compared with the unmodified CBD. Preferably, at least 1, preferably at least 2, more preferably at least 3, 4 or 5, and yet more preferably at least 10 amino acids of a CBD are modified. Modifications can be made to a number of regions.

In a preferred embodiment, a solvent exposed region is modified and, preferably, a number of such regions are modified. Preferred solvent exposed regions are the loops of the CDB. Suitably, modifications are made to alter the size and/or area of a loop, preferably to increase the size and/or area of the loop. The size may suitably be increased by at least 1, 2, 3, 4 or 5 amino acids and preferably by at least 10 or 20 amino acids. A loop size may be increased by up to as many as 40, or even maybe as many as 50 amino acid residues. Modifications can be made to any of the L1, L2, L3, L4, L5, L6 and L7 loops as defined by IL-6R and/or FIG. 5. Suitably, modifications are made to at least two or three different solvent exposed regions, e.g. to at least two or three of any the L1, L2, L3, L4, L5, L6 and L7 loops. The solvent exposed regions can be modified by insertion, substitution or by other suitable modifications described herein.

For example, loop L1 in IL-6R is positioned in the centre of the CBD (FIGS. 1, 2, 4 and 6). Since loop L1 of the CBD of IL-6R contains a natural disulphide bond, this might constrain the flexibility and so form an ideal semi-rigid scaffold for the display of larger, protruding ‘finger-like’ loops by insertion of additional amino acids within the L1 loop. These protruding ‘finger-like’ loops are then likely to provide a complementary binding surface to cavities within the target antigen (protein) to which the CBD is capable of binding, analogous to the protruding loops observed in natural camelid VhH and shark NAR domains (Muyldermans S et al., 2001 Trends Biochem Sci. 26(4):230-5) and (Nuttall S D et al., 2000 Curr Pharm Biotechnol. 1(3):253-63).

Also encompassed are modifications which are essentially tantamount to conservative substitutions throughout the sequence but which alter a property of the CBD. Such conservative substitutions are shown in Table 3.

TABLE 3
Exemplary conservative substitutions.

| Original Residue | Exemplary Substitutions |
|---|---|
| Ala (A) | val; leu; ile; gly |
| Arg (R) | lys |
| Asn (N) | gln; his; |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe; |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Furthermore, if desired, non-naturally occurring amino acids or chemical amino acid analogues can be introduced as a substitution or addition into the polypeptide of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also provided by the invention are chemically modified derivatives of CBDs which may provide advantages such as increasing stability and circulating time of the polypeptide, or decreasing immunogenicity (see U.S. Pat. No. 4,179,337). The chemical moieties for derivitization may be selected from water-soluble polymers such as polyethylene glycol, ethylene glycol/propylene glycol copolymers, carboxymethylcellulose, dextran, polyvinyl alcohol and the like.

Also included are binding moieties which are differentially modified during or after synthesis, e.g., by biotinylation, benzylation, glycosylation, acetylation, phosphorylation, amidation, derivatization by known protecting/blocking groups, proteolytic cleavage, etc. The CBDs may be modified at random positions within the molecule, or at predetermined positions within the molecule and may include one, two, three or more attached chemical moieties. These modifications may, for example, serve to increase the stability and/or bioactivity of the binding moieties of the invention.

The CBDs may also be modified by having carboxy-terminal truncations. However, the scope for such modifications is limited and it is preferred that no more than 8 residues, more preferably not more than 6 residues, of the last beta strand in the FnIII-like domains is removed. Preferably, there is no truncation in the first FnIII-like domain.

### Altering Binding Characteristics

In a preferred embodiment, the modification alters the binding characteristics of the CBD. The cytokine binding region which normally contacts the natural ligand for the CBD is typically the solvent exposed region of the CBD and is generally made up of the surface exposed loops. For example, domains D2 and D3 of IL-6R together comprise 7 cytokine binding loops (L1 to L7), as described above. The location of these loops in other CBDs is shown in FIG. 5. Thus it is preferred that modifications are made to one or more of these loop regions, or the equivalent regions in other CBDs, in order to alter the binding characteristics.

For example, the binding affinity of the CBD for at least one of its natural ligands can be reduced or abolished. Preferably at least a two-fold, more preferably at least a five or ten-fold reduction in binding affinity for at least one natural ligand is achieved.

In one embodiment, the binding specificity of the modified CBD is different to that of the unmodified CBD. Preferably, the unmodified CBD is derived from the extracellular domain of a first receptor having specificity for a first ligand and one or more of the loops of the unmodified CBD having been replaced with the corresponding one or more loops from a second receptor having specificity for a second ligand with the result that the modified CBD has a specificity for the second ligand. For example, the binding specificity of the CBD could be altered to a different cytokine. In particular, this can be achieved by replacing the loops in the cytokine binding region of a CBD which has specificity for a first cytokine, with the loops from a cytokine binding region of a second CBD which has specificity for a second cytokine. For example, the loops L1 to L7 of the CBD of IL-6R could be replaced by loops L1 to L7 of the CBD of IL-11R to provide the modified binding moiety with specificity for IL-11 instead of IL-6. Similarly, the loops L1 to L7 of the CBD of IL-6R could be replaced by loops L1 to L7 of the CBD of prolactin receptor, LIF receptor or oncostatin M receptor to provide the modified binding moiety with specificity for prolactin and/or growth hormone, LIF or oncostatin M respectively instead of IL-6.

In a preferred embodiment, the first receptor is the IL-6R and the second receptor is either prolactin receptor, LIF receptor or oncostatin M receptor, thus altering the ligand specificity of the CBD from IL-6 to either prolactin and/or growth hormone or LIF or oncostatin M, respectively.

In an alternative preferred embodiment, the first CBD is prolactin receptor, or IL-11R, or CNTF receptor which has been modified such that the loops of the cytokine binding region have been replaced with the loops of a second cytokine receptor region alters the specificity of the first CBD.

Modifications can also be made to regions of the CBD that are not solvent exposed and/or which do not form part of a cytokine binding loop (i.e. L1 to L7). For example, the binding moiety may comprise one or more modifications to the hinge region of the CBD and/or to the binding interface of the FnIII-like domains of the CBD. Modifications to the binding interface between the two FnIII-like domains may result in an altered geometry of the spatial relationship between the two domains. This in turn can be used to alter the orientation and/or association of the solvent exposed binding regions, e.g. the loops, which will modify the characteristics/topology of the overall binding surface.

Modifications to the binding interface between the two FnIII-like domains may, for example, involve modifying, either directly or indirectly (e.g. sterically), generally highly conserved hydrophobic residues which are buried and which act to stabilise the association between the two domains. For example, it may involve modifying one or more of residues Pro107, Leu195 and Pro197 of D2 of IL-6R and Trp225, Leu232, Ala275, Pro200 and Pro222 of D3 of IL-6R, or corresponding residues in other CBDs.

### Altering Physicochemical Properties

In a preferred embodiment, a modification alters, and preferably improves, the biophysical and/or physicochemical properties of the binding moiety. Preferably, the modification alters, preferably improves, the stability and/or solubility properties of the binding moiety.

For example, modifications at the domain interface, including interface mutations, can be made to improve surface complementarity. For example, cysteine residue insertions may be made to provide for disulphide stabilisation.

Modifications may also be made to alter, preferably improve, the stability of the scaffold structure. For example, amino acids may be substituted with other amino acids having larger side chains in order to fill out internal holes in the globular structure. Such substitutions could include, for example, glycine to alanine, asparagine to glutamine, aspartate to glutamate, phenylalanine to tyrosine or tryptophan, tyrosine to tryptophan, asparagine or aspartate to histidine, histidine to tyrosine and lysine to arginine. Glycine residues may also be substituted to decrease the flexibility of the protein backbone. In contrast, Proline residues may be inserted or substituted to improve the flexibility of the scaffold, e.g. where there are limitations in the dihedral angles of the protein backbone and in the secondary structure. Other suitable modifications for altering, and in particular improving stability, will be apparent to the skilled person.

In a preferred embodiment, the binding moiety is modified so as to alter, and preferably improve, its solubility as compared with the unmodified binding moiety. A variety of strategies may be employed to improve solubility and in particular design binding moieties that are solubly expressible in cellular hosts (i.e. non-aggregating). For example, modifications can be made that (i) reduce hydrophobicity by replacing solvent exposed hydrophobic residues with suitable polar residues; (ii) increase polar character by replacing neutral polar residues with charged polar residues; (iii) replace non-disulphide bonded cysteine residues (unpaired cysteines) with suitable non-cysteine residues, and (4) replace residues whose identity is different in the corresponding CBD derived from another species (e.g. substitute murine IL-6R residues into human IL-6R). Other alternative strategies will also be apparent to the skilled person. For example, modifications that increase the stability of a protein can sometimes improve solubility by decreasing the population of partially folded or misfolded states. As another example, protein solubility is typically at a minimum when the isoelectric point of the protein is equal to the pH of the surrounding solution. Modifications, which perturb the isoelectric point of the protein away from the pH of a relevant environment, such as serum, can therefore serve to improve solubility.

In a preferred embodiment, one or more, preferably hydrophobic, residues in solvent exposed regions, preferably in a loop, are replaced with structurally and functionally compatible polar residues. Alanine and glycine may also serve as suitable replacements, constituting a reduction in hydrophobicity.

In an alternate embodiment, preferred polar residues include those that are observed at homologous positions in other CBDs.

In another preferred embodiment, free cysteine residues (that is, cysteine residues that are not participating in disulphide bonds) are mutated to a structurally and functionally compatible non-cysteine residue. Unpaired cysteines can be identified by visual analysis of the structure or by analysis of the disulphide bond patterns of related proteins.

In a preferred embodiment, if the non-disulphide forming cysteine position is substantially buried in the CBD framework, the cysteine may be removed or replaced with, for example, a suitable non-cysteine residue such as alanine or serine. If the cysteine position is substantially exposed to solvent, suitable non-cysteine residues include alanine and the polar residues. Furthermore, cysteine residues not involved in disulphide bond formation within the CBD framework could also be removed or replaced, e.g. with alanines or serines, so as to improve solubility. For example, as regards D2 and D3 of the IL-6R CBD, any one or more of Cys174, Cys192 and Cys258 could be removed, and preferably replaced with serines, to improve solubility.

In a preferred embodiment, one or more solvent exposed loops is/are modified to improve solubility. Solubility may be improved by, for example, either removing disulphide bondforming cysteines and/or replacing disulphide bond-forming cysteines from within the solvent exposed loops with amino acids such as alanine or serine.

Modifications to improve solubility may be desirable where the binding moieties are being designed to function in an intracellular context and/or their method of production favours expression in a soluble form. It will also be evident to the skilled person that it may be necessary to modify the solubility characteristics of the binding moiety at the same time or even prior to making other modifications, such as, changing the binding characteristics.

The physicochemical properties, such as stability and solubility, of the binding moieties may be qualitatively and/or quantitatively determined using a wide range of methods known in the art. Methods which may find use in the present invention for characterizing the biophysical/physicochemical properties of the binding moieties include gel electrophoresis, chromatography such as size exclusion chromatography, reversed-phase high performance liquid chromatography, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use.

For example, protein stability (e.g. structural integrity) may be determined by measuring the thermodynamic equilibrium between folded and unfolded states.

In one embodiment, stability and/or solubility may be measured by determining the amount of soluble protein after some defined period of time. In such an assay, the protein may or may not be exposed to some extreme condition, for example elevated temperature, low pH, or the presence of denaturant. Because unfolded and aggregated protein is not expected to maintain its function, e.g. be capable of binding to a predetermined target molecule, the amount of activity remaining provides a measure of the binding moieties stability and solubility. Thus, one method of assessing solubility and/or stability is to assay a solution comprising a binding moiety for its ability to bind a target molecule, then expose the solution to elevated temperature for one or more defined periods of time, then assay for antigen binding again.

Alternatively, the modified binding moieties could be expressed in prokaryotic expression systems and the protein isolated from the cell lysate by a series of biochemical purification steps including differential centrifugation, affinity isolation chromatography using attached tags such as poly histidine, ion-exchange chromatography and gel filtration chromatography. A measure of the improvement in the solubility of the modified polypeptide can be obtained by making a comparison of the amount of soluble protein obtained at the end of the purification procedure to that obtained using the unmodified polypeptide, when starting with a similar amount of expressed unfractionated product. Levels of expression of product in culture can be normalised by a comparison of product band densities after polyacrylamide gel electrophoresis of equivalent aliquots of SDS detergent-solubilised cell lysate.

Alternatively, binding moieties can be unfolded using chemical denaturant, heat, or pH, and this transition be monitored using methods including, but not limited to, circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques.

The solubility of the binding moieties of the present invention preferably correlates with the production of correctly folded, monomeric polypeptide. The solubility of the modified binding moiety may therefore also be assessed by HPLC or FPLC, using which soluble (non-aggregated) fragments will give rise to a single peak, whereas aggregated fragments will give rise to a plurality of peaks. A preferred measurement of solubility uses conventional FPLC or HPLC techniques which assess the level of aggregation and presence of high molecular weight species as described in Power B E et al., 2003, Protein Science 12, 734-747.

As an example of an accelerated stability trial, aliquots of the binding moiety can be stored at different temperatures, such as −20° C., 4° C., 20° C. and 37° C. and the activity of the binding moiety assayed at different time intervals. For example, successful maintenance of activity during storage at 37° C. for 12 weeks is roughly equivalent to storage stability for 12 months at 4° C. The trial can also be conducted to compare the effect of different protecting additives in the storage buffer on the stability of the protein. Such additives can include compounds such as glycerol, sorbitol, non-specific protein such as bovine serum albumin, or other protectants that might be used to increase the shelf life of the protein.

In a preferred embodiment, cysteine residues have been removed or replaced within the CBD, preferably from within one or more of the loops. In a further preferred embodiment, cysteine residues have been removed or replaced in one or more loops of one FnIII-like domain whilst remaining unaltered in the other FnIII-like domain.

It will be understood that any one or more of the type of modifications described above in relation to altering a particular property of a binding moiety may be used to alter other properties in addition to or instead of those which are specifically described in relation to that modification above.

Binding moieties of the invention may be in a substantially isolated form. It will be understood that the protein may be mixed with carriers or diluents which will not interfere with the intended purpose of the protein and still be regarded as substantially isolated. Binding moieties of the invention may also be in a substantially purified form, in which case they will generally comprise the protein in a preparation in which more than 90%, e.g. 95%, 98% or 99% of the protein in the preparation is a binding moiety of the invention.

The binding moieties of the invention may be linked to other molecules, for example by covalent or non-covalent means. In preferred embodiments, the binding moieties (CBD) of the invention may be linked (without restriction) to molecules such as enzymes, drugs, lipids, sugars, nucleic acids and viruses.

In one embodiment, the binding moiety may contain solvent exposed cysteine residues for the site-specific attachment of other entities.

Binding moieties of the invention can be linked to other molecules, typically by covalent or non-covalent means. For example, binding moieties may be produced as fusion proteins, linked to other polypeptide sequences. Fusion partners can include enzymes, detectable labels and/or affinity tags for numerous diagnostic applications or to aid in purification. Fusion partners, without restriction, may be GFP (green fluorescent protein), GST (glutathione S-transferase), thioredoxin or hexahistidine. Other fusion partners include targeting sequences that direct binding moieties to particular sub-cellular locations or direct binding moieties to extracellular locations e.g. secretion signals. In a preferred embodiment binding moieties of the invention do not comprise other regions of the receptor/protein from which they are derived i.e. any fusion partners are heterologous to the CBD. The heterologous sequence may be any sequence which allows the resulting fusion protein to retain the activity of the modified CBD. The heterologous sequences include for example, immunoglobulin fusions, such as Fc fusions, or fusions to other cellular ligands which may increase stability or aid in purification of the protein.

Diagnostic or therapeutic agents that can be linked to the binding moieties of the invention include pharmacologically active substances such as toxins or prodrugs, immunomodulatory agents, nucleic acids, such as inhibitory nucleic acids or nucleic acids encoding polypeptides, molecules that enhance the in vivo stability or lipophilic behaviour of the binding moieties such as PEG, and detectable labels such as radioactive compounds, dyes, chromophores, fluorophores or other imaging reagents.

Binding moieties may also be immobilised to a solid phase, such as a substantially planar surface (e.g. a chip or a microtitre plate) or beads. Techniques for immobilising polypeptides to a solid phase are known in the art. In addition, where libraries of binding moieties are used (e.g. in screening methods), arrays of binding moieties immobilised to a solid phase can be produced (Lee Y S and Mrksich, M, 2002 Trends Biotechnol. 20(12 Suppl):S14-8. and references contained therein).

In another embodiment of the invention, the binding moieties of the invention function as a protein scaffold with other polypeptide sequences being inserted into solvent-exposed regions of the binding moiety for display on the surface of the scaffold. Such scaffolds may, for example, serve as a convenient means to present peptides in a conformationally constrained manner. The scaffolds may be used to produce CBDs with altered binding specificities and also to produce and/or screen for binding moieties having specificity for any target molecule of interest.

Heterologous polypeptide sequences may be inserted into one or more solvent exposed regions such as, for example, one or more loops of the CBD. The CBD of the binding moiety functions as a protein scaffold for the inserted heterologous sequences, displaying the heterologous sequences on the surface of the binding moiety.

The heterologous sequences may replace all or part of the loop of the CBD into which they are inserted, or may simply form additional sequence. Preferably, a plurality of heterologous sequences are inserted into a plurality of loops.

The heterologous sequences may be derived from solvent exposed regions such as, for example, loops of another CBD. They may also be derived from other non-CBD molecules or be partially of fully randomised.

Other modifications can also be made to the scaffold proteins of the invention as described in the previous sections in relation to CBDs and they may also be linked to other molecules and/or produced as multimers as described below.

Two or more CBDs may be joined together to form multimers through either covalent linkage or non-covalent linkage or a combination of linkages, including the use of chemical or genetically-encoded linkers. CBD multimers are one preferred design for therapeutic reagents since they have the potential to provide increased avidity and slower blood clearance rates which may provide favourable pharmacokinetic and biodistribution properties. The linkages used are well known to persons skilled in the art, for example in relation to antibodies and antibody fragments joined by chemicals (Casey J L et al., 2002 Br J Cancer. 86(9):1401-10), linkages is by way of genetically-encoded linker polypeptides (BITE's scFv-scFv), or adhesive fusion-domains (Pluckthun, A., and Pack, P 1997. Immunotechnology 3, 83-105). Indeed, two FnIII-like domains from different CBDs may be cross-paired using linker polypeptides to form tightly-associated CBD multimers in the manner of a diabody (an antibody Fv dimer) or triabody (antibody Fv trimer) or tetrabody (antibody Fv tetramer) (Power B E et al., 2001, Cancer Immunol Immunother. 50(5):241-50). The resulting CBD multimers from any of these linker strategies described above may possess the same, or different target specificities thus providing multivalent or multispecific reagents. In a preferred embodiment, two CBDs may be joined to form a dimer through either covalent linkage or non-covalent linkage or a combination of linkages thereby providing two target binding affinities. If two or more CBDs in the multimer have the same target specificity, the CBD multimer will be multivalent and have increased avidity (functional affinity) for binding to two or more target molecules.

CBD multimers may be designed to have increased stability by modification to the interface contact regions, either through chemical or genetic alterations. For example, detailed examination of the CBD framework regions at the multimer interface may direct introduction of residue mutations or chemical modifications that stabilise the interface and thereby direct the preferential formation of CBD multimers. In one embodiment, the mutations are introduced to interface residues other than F134(D2), F168 (D2) and H261 (D3). In another embodiment, the mutation is introduced at residue C174 (D2), C192 (D2) or C258 (D3).

### Production of Binding Moieties

Binding moieties of the invention may be made by chemical or recombinant means. Techniques for chemically synthesising peptides are reviewed by Borgia and Fields, 2000, TibTech 18: 243-251 and described in detail in the references contained therein. Typically binding moieties of the invention are made by recombinant means. Accordingly, the present invention provides polynucleotides encoding binding moieties of the present invention.

Modifications to binding moieties of the invention can be made using standard cloning techniques known to persons skilled in the art, such as site-directed mutagenesis. Variation in the amino acid sequence of a natural unmodified loop or loops can be achieved by designing the encoding gene to produce either specific point mutations or by random ‘window’ mutagenesis to randomise the entire loop sequence(s) during the construction of a library repertoire. Variation in loop length may be achieved by designing the encoding gene to remove some of the amino acids in the CBD loops, thus making shorter loops or conversely by increasing the number of amino acids to extend the loops. These designs can be applied to two or more loops selected from L1, L2, L3, L4, L5, L6 and L7 loops. Alternatively the entire gene repertoire comprising the CBD framework and the randomised loops can be constructed using synthetic oligonucleotide primers.

One approach to obtaining binding moieties having a binding affinity for a target molecule of interest is to produce libraries of polynucleotides which encode different binding moieties of the invention comprising modifications in the CBR, preferably in one or more loops, and screen the libraries for binding to the target molecule using standard techniques such as phage display or ribosomal display. This screening approach will be described in more detail below.

Polynucleotides, Vectors and Hosts

Polynucleotides of the invention may comprise DNA or RNA. They may be single-stranded or double-stranded. They may also be polynucleotides which include within them synthetic or modified nucleotides. A number of different types of modifications to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

Polynucleotides of the invention can be incorporated into a recombinant replicable vector. The vector may be used to replicate the nucleic acid in a compatible host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by a host cell or using an in vitro transcription/translation system, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell to provide for expression of a binding moiety of the invention. This process may comprise culturing a host cell transformed with an expression vector under conditions to provide for expression by the vector of a coding sequence encoding the binding moiety, and optionally recovering the expressed binding moiety.

The vectors may be, for example, plasmid, phagemid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in prokaryotic or eukaryotic cells. With respect to eukaryotic promoters, they may be promoters that function in a ubiquitous manner or, alternatively, a tissue-specific manner. They may also be promoters that respond to specific stimuli. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the binding moiety can be regulated during the life-time of the cell. Inducible means that the levels of expression obtained using the promoter can be regulated.

In a number of embodiments of the present invention, heterologous sequences are inserted into the binding moieties of the present invention, for example where the binding moieties are used as scaffold sequences. Such modifications are generally made by manipulating polynucleotides of the invention encoding binding moieties of the invention. This may conveniently be achieved by providing cloning vectors that comprise a sequence encoding a CBD which sequence comprises one or more unique insertion sites in one or more regions encoding a solvent exposed region of said cytokine domain, to allow for easy insertion of nucleotide sequences encoding heterologous sequences into the appropriate regions of the CBD.

Each “unique” insertion site typically contains a nucleotide sequence that is recognised and cleaved by a type II restriction endonuclease, the nucleotide sequence not being present elsewhere in the cloning vector such that the cloning vector is cleaved by the restriction endonuclease only at the “unique” insertion site. This allows for easy insertion of nucleotide sequences having the appropriate ends by ligation with cut vector using standard techniques well know by persons skilled in the art. Preferably the insertion site is engineered—i.e. where the CBD is derived from a naturally occurring sequence, the insertion site does not naturally occur in the natural sequence.

Vectors and polynucleotides of the invention may be introduced into host cells for the purpose of replicating the vectors/polynucleotides and/or expressing the binding moiety proteins of the invention encoded by the polynucleotides of the invention. Host cells include prokaryotic cells such as bacterial cells and eukaryotic cells including yeast, fungi, insect cells and mammalian cells.

Vectors/polynucleotides of the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

Host cells comprising polynucleotides of the invention may be used to express proteins of the invention. Host cells are cultured under suitable conditions which allow for expression of the binding moieties of the invention. Expression of the binding moieties may be constitutive such that they are continually produced, or inducible, requiring a stimulus to initiate expression. In the case of inducible expression, protein production can be initiated when required by, for example, addition of an inducer substance to the culture medium, for example dexamethasone or IPTG, or inducible expression may achieved through heat-induction, thereby denaturing the repressor and initiating protein synthesis.

Binding moieties of the invention can be extracted from host cells by a variety of techniques known in the art, including enzymatic, chemical and/or osmotic lysis and physical disruption.

### Libraries of Binding Moieties

Binding moieties of the present invention may be provided as libraries comprising a plurality of binding moieties which have different sequences in the CBR. Preferably, the variations reside in one or more loops. These libraries can typically be used in screening methods to identify a binding reagent with an activity of interest, such as affinity for a specific target molecule of interest.

Libraries of binding moieties are conveniently provided as libraries of polynucleotides encoding the binding moieties. The polynucleotides are generally mutagenised or randomised to produce a large number of different sequences which differ at one or more positions within at least one loop.

Mutations can be introduced using a variety of techniques known in the art, such as site-directed mutagenesis. A number of methods for site-directed mutagenesis are known in the art, from methods employing single-stranded phage such as M13 to PCR-based techniques (see “PCR Protocols: A guide to methods and applications”, M. A. Innis, D. H. Gelfand, J. J. Sninsky, T. J. White (eds.). Academic Press, New York, 1990). Another technique is to use the commercially available “Altered Sites II in vitro Mutagenesis System” (Promega—U.S. Pat. No. 5,955,363). Techniques for site-directed mutagenesis are described above. Pluralities of randomly mutated sequences can be made by introducing mutations into a nucleotide sequence or pool of nucleotide sequences ‘randomly’ by a variety of techniques in vivo, including; using ‘mutator strains’, of bacteria such as E. coli mutD5 (Low et al., 1996, J Mol Biol 60: 9-68); and using the antibody hypermutation system of B-lymphocytes (Yelamos et al., 1995, Nature 376: 225-9). Random mutations can also be introduced both in vivo and in vitro by chemical mutagens, and ionising or UV irradiation (Friedberg et al., 1995, DNA repair and mutagenesis. SM Press, Washington D.C.), or incorporation of mutagenic base analogues (Zaccolo et al., 1996 J Mol Biol 255: 589-603). ‘Random’ mutations can also be introduced into genes in vitro during polymerisation for example by using error-prone polymerases (Leung et al., 1989, Technique 1: 11-15).

It is generally preferred to use mutagenesis techniques that vary the sequences present in the cytokine binding region (e.g. the loop sequences) of the CBD, although framework changes may also occur which may or may not be desirable. One method for targeting the cytokine binding region is to provide a plurality of relatively short nucleotide sequences that are partially or fully mutagenised/randomised and clone these sequences into specific insertion sites in the binding moiety, as described above in relation to scaffold sequences.

Another approach is to synthesise a plurality of random synthetic oligonucleotides and then insert the oligonucleotides into a sequence encoding the binding moiety and/or replace a sequence encoding the binding moiety with the random synthetic oligonucleotides. A suitable method is described in WO97/27213 where degenerate oligonucleotides are produced by adding more than one nucleotide precursor to the reaction at each step. The advantage of this method is that there is complete control over the extent to which each nucleotide position is held constant or randomised. Furthermore, if only C, G or T are allowed at the third base of each codon, the likelihood of producing premature stop codons is significantly reduced since two of the three stop codons have an A at this position (TAA and TGA).

Another approach is to generate the gene repertoire using SOE-PCR (splicing overlap extension polymerase chain reaction) a method known to those in the art. This method is used when no full length gene template is available and the gene repertoire is synthetically assembled.

Oligonucleotide synthesis is performed using techniques that are well known in the art (see Eckstein, Oligonucleotides and Analogues: A Practical Approach, IRL Press at Oxford University Press 1991). Libraries can also be specified and purchased commercially. The synthetic process can be performed to allow the generation of all or most possible combinations over the length of the nucleic acid, thus generating a library of randomised nucleic acids. These randomised sequences are synthesised such that they allow in frame expression of the randomised peptide with any fusion partner.

In one embodiment, the library is fully randomised, with no sequence preferences or constants at any position. In another embodiment, the library is biased, i.e. partially randomised in which some positions within the sequence are either held constant, or are selected from a limited number of possible variations. Thus some nucleic acid or amino acid positions are kept constant with a view to maintaining certain structural or chemical characteristics.

The randomised oligonucleotides can then be inserted into a suitable site and/or replace a suitable sequence encoding a binding moiety.

Generally the library of sequences will be large enough such that a structurally diverse population of random sequences is presented. This ensures that a large subset of 3-D shapes and structures is represented and maximises the probability of a functional interaction.

It is preferred that the library comprises at least 1000 different nucleotide sequences, more preferably at least 10⁴, 10⁵ or 10⁶ different sequences. Preferably, the library comprises from 10⁴ to 10¹⁰ different sequences. Preferably at least 5, 10, 15 or 20 amino acid residues of the peptides encoded by the nucleotide sequences are randomised.

Typically, the inserted peptides encoded by the randomised nucleotide sequences comprise at least 5, 8, 10 or 20 amino acids. Preferably, they also comprise fewer than 50, 30 or 25 amino acids.

The libraries of polynucleotides encoding binding moieties can be screening using any suitable technique to identify a binding moiety having an activity of interest. For example, to identify a binding moiety that binds to a target molecule of interest, the library of polynucleotides is incubated under conditions that allow for expression of the binding moiety polypeptides encoded by the polynucleotides and binding of the polypeptides to the target molecule assessed. Binding is typically assessed in vitro or using whole cell assays.

Suitable techniques for screening the library for binding moieties having an activity of interest include phage display and ribosome display as well as the use of viral vectors, such as retroviral vectors.

The sequence of binding moieties identified in the screen can conveniently be determined using standard DNA sequencing techniques.

### Diagnostic/Therapeutic Uses of Binding Moieties

Binding moieties of the invention, including those identified in the screening methods of the invention, may be used in methods of diagnosis/therapy by virtue of their specific binding to a target molecule of interest. Such uses will be analogous to the plethora of diagnostic/therapeutic applications already known in relation to antibodies and fragments thereof. For example, binding moieties of the invention may be used to detect the presence or absence of molecules of interest in a biological sample.

For diagnostic purposes, it may be convenient to immobilise the binding reagent to a solid phase, such as a dipstick, microtitre plate or chip.

As discussed above, binding moieties of the invention when used diagnostically will typically be linked to a diagnostic reagent such as a detectable label to allow easy detection of binding events in vitro or in vivo. Suitable labels include radioisotopes, dye markers or other imaging reagents for in vivo detection and/or localisation of target molecules.

Binding moieties may also be used therapeutically. For example, binding moieties may be used to target ligands that bind to extracellular receptors, such as cytokine receptors, and consequently antagonise the effect of such ligands. Cytokines and their receptors are involved in a wide range of disease processes and consequently modulation of their activity with specifically designed binding moieties based on CBDs has clear clinical implications.

In addition, binding moieties of the invention may be used, in a similar manner to antibodies, to target pharmacologically active substances to a cell of interest, such as a tumour cell, by virtue of binding to a cell surface molecule present specifically on the tumour cell to which the binding moiety binds specifically.

### Administration

Binding moieties of the invention including binding moieties identified by the screening methods of the invention may preferably be combined with various components to produce compositions of the invention. Preferably the compositions are combined with a pharmaceutically acceptable carrier, adjuvant or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular, oral or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding binding moieties may be administered directly as a naked nucleic acid construct. When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example Lipofectam™ and Transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, oral, intraocular or transdermal administration.

The routes of administration and dosages described are intended only as a guide since a skilled practitioner will be able to determine readily the optimum route of administration and dosage for any particular patient and condition.

The various features and embodiments of the present invention, referred to in individual sections above apply, as appropriate, to other sections, mutatis mutandis. Consequently features specified in one section may be combined with features specified in other sections, as appropriate.

### EXAMPLE 1

### Design of Modified IL-6R CBD with Altered Binding Specificity

A PSI_BLAST search of the Brookhaven protein data bank revealed several structures that are closely related to the cytokine binding modules of the human IL-6 receptor. Of these the human prolactin receptor (PRLR) bound to human growth hormone was the most closely related structure that did not have overlapping specificity for interleukin-6. The binding of human growth hormone by the prolactin receptor is mediated by the same loop framework as the cytokine binding modules of IL-6R use to bind IL-6.

### Sequence Alignment

The sequences of IL-6R and PRLR have been aligned according to their three dimensional structure using the MALIGN3D function of MODELLER6v2.

### Loop Definition

Residues from the prolactin receptor in contact with human growth hormone were selected using VMD. VMD is a visualisation package developed at the University of Illinois which allows the viewing and manipulation of large molecules (Schwieters (2001) Journal of Magnetic Resonance 149:239-244). Loop regions were selected to contain these residues and residues which support the correct side-chain orientation of the contact residues.

### Homology Modelling

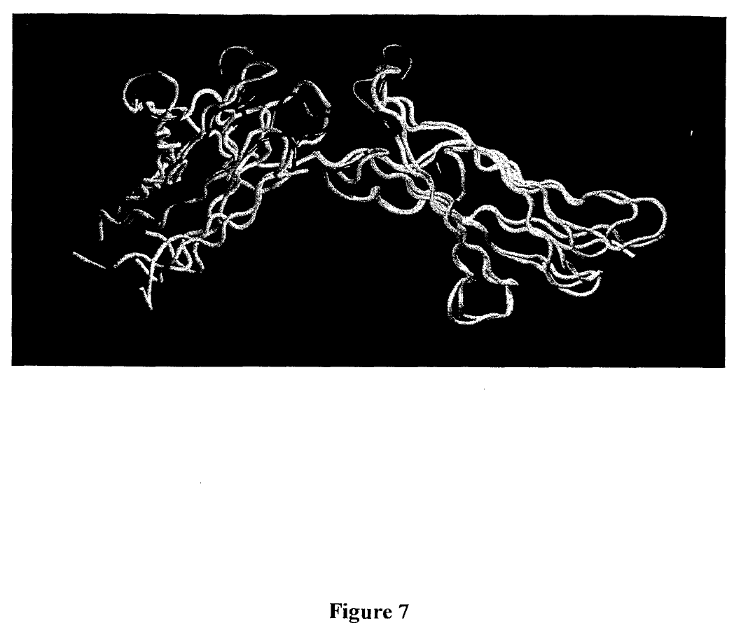

The sequence of a CBD binding moiety protein incorporating the framework residues of IL-6R and loop residues from the prolactin receptor was created. An initial series of homology models of the CBD binding moiety was generated using MODELLER6v2 with IL-6R framework residues and prolactin receptor loop residues as templates (see FIG. 7). Model quality was assessed using PROCHECK. The loop regions were then refined ab initio using MODELLER6v2. Final model was then energy minimised and assessed for stability using CNS (Brünger A T et al., 1998 Acta Crystallog D54:905-921).

### EXAMPLE 2

### Production of an IL-6R CBD (Binding Moiety)

Oligonucleotide primers were designed to amplify the CBD domains (the D2 and D3 domains) of human IL-6R by PCR, using IL-6R DNA as a template for this reaction. These PCR fragments of correct size and DNA sequence were cloned into pPOW5 bacterial expression vector. Protein expression was performed using eight different bacterial cell strains. One particular strain was selected for further stability and characterisation studies.

### EXAMPLE 3

### Modification of an IL-6R CBD to Introduce Prolactin Binding Specificity

In another gene construct, the surface loops of prolactin receptor were grafted onto the IL-6R framework to produce a reagent with prolactin binding specificity. The grafting process involved replacement of seven solvent-exposed surface loops L1 to L7 of IL-6R by the equivalent loop residues from prolactin receptor, thereby effectively changing the binding specificity of the modified CBD from IL-6 to prolactin. There are several methods that can result in loop grafting and, in this example, the grafting process involved redesigning the gene encoding the modified IL-6R CBD such that the encoded surface loops L1 to L7 were that of prolactin receptor. The modified CBD gene was then constructed using a gene assembly process using synthetic oligonucleotides, typically 80 bases in length, which were assembled by hybridisation and ligation, into a section of double-stranded DNA encoding the entire modified CBD gene, in an overlapping “brick-laying” fashion. PCR and oligonucleotide primers were used as the final step to amplify the fully assembled gene. The DNA sequence of the PCR product was confirmed, and the modified CBD gene then sub-cloned and expressed in bacteria.

### EXAMPLE 4

### Producing a Novel Binding Moiety with Modified Intra-Domain Disulphide Bonds

We produced a binding moiety with a modified intra-domain disulphide bond. We used PCR to introduce a mutation at Cys174 to Ser on the CBD framework. This Cys174 in D2, usually forms a disulphide bond with another cysteine in the first domain of IL-6R (a non-FnIII domain commonly referred to as the D1 domain of IL-6R), and is not involved with the D2 and D3 CBD associations. The Cys174→Ser mutant was subsequently expressed in bacteria.

### EXAMPLE 5

### Producing a Novel Binding Moiety with No Cysteine Residues in the D3 Domain

We introduced another CBD framework mutation Cys258 to Serine in domain D3. This is a buried cysteine residue, mutated in an attempt to increase expression and stability of the CBD framework, and to ascertain whether the D3 domain could fold without the need for this Cysteine residue. We have expressed the CBD containing this D3 mutation in bacteria.

Clones isolated from the D3 library also contained this Cys258→to Ser framework mutation (see Examples 7 and 8).

### EXAMPLE 6

### Producing a Novel Binding Moiety with a Removed (Replaced) Cysteine Residues in the Solvent Exposed Region

We noticed that when the PRLR loop graft onto the IL-6R framework was expressed in bacteria, there were less protein aggregates. There is a solvent exposed Cys 192 in the IL-6R framework/loop junction, that is not involved in disulphide bond formation, which is not a cysteine residue in the equivalent position of the PRLR loop. Another mutation Cys192→Ser, which lies at this framework/loop junction was designed within the D2 domain of IL-6R. This is a solvent exposed cysteine in the IL-6R framework and this mutation improved solubility of the IL-6R framework CBD.

### EXAMPLE 7

### Producing a Library Repertoire of Novel Binding Moieties Based on the CBD Scaffold

A gene library comprising the IL-6R CBD was constructed with mutations in the solvent-exposed surface loops. Loops L5, L6 and L7 were mutated in the D3 domain of the CBD by constructing a gene repertoire using overlapping synthetic oligonucleotides and the gene assembly techniques described in Example 3. The overlapping oligonucleotides contained flanking framework residues of IL-6R, then genetic diversity in the loops residues, followed by more framework residues. The genetic diversity encoding the amino acid residues in the loops was biased in such a way as to reduce the chance of stop codons and also to encode for all 20 amino acids at each position of each loop. This diversity was achieved during the synthesis of the degenerate oligonucleotides, wherein instead of adding one nucleotide per position at a time, all four nucleotides (G, A, T and C) were added per position. Stop codons triplets usually end with an A e.g. TAA. The chance of this occurring in the degenerate oligonucleotide was reduced by only allowing G, T and C at the third position of the triplet.

In order to make the genetically diverse library, two different lengths of oligonucleotides were used. The oligonucleotides covering the loop regions were about 80 bases in length (top strand). The reverse oligonucleotide “cementing the bricks” were short, covering only the framework residues, and were about 55 bases in length. PCR was used to fill-in the gaps on the bottom strand. The cloned gene repertoire in the phagemid vector was transformed into bacterial competent cells. Several well-spaced isolated colonies were picked and grown in liquid culture, from which the DNA was extracted and sequenced. The DNA sequence from one of these isolated clones showed mutations within both loop regions as well as the CDB framework.

The IL-6R CBD library framework contained three mutations in which cysteine residues (Cys174, Cysr192 and Cys258) had been replaced by serine residues. In addition to the desired framework changes, the DNA sequence showed changes in loop 6, with residues in that loop being replaced with other residues. This clone was subsequently expressed in bacteria.

The partial DNA sequence of IL-6R D3 (loops 6 and 7 in bold and boxed, and Cys258 in bold) is shown below as sequence (a). The corresponding partial DNA sequence of the D3 library clone, showing changes in loop 6 and at Cys258 (mutated to Ser) shown as sequence (b).

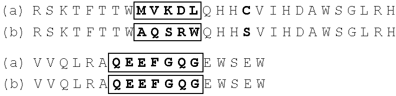

### EXAMPLE 8

### Producing a Novel Binding Moiety with Multi-Loop Mutations

Another clone isolated from the D3 library described in Example 7 showed changes in both loop 6 and loop 7 residues of the D3 domain. This clone, also containing a CBD framework mutation at Cys258 to Ser, was also expressed in bacteria.

The partial DNA sequence of IL-6R D3 (loops 6 and 7 in bold and boxed, and Cys258 in bold) is shown below as sequence (c). The corresponding partial DNA sequence of the D3 library clone, showing changes in loops 6 and 7 and at Cys258 (mutated to Ser) shown as sequence (d).

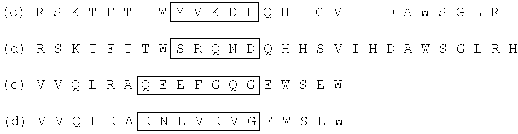

Examples 1 to 8 demonstrate that a functional CBD scaffold can be made from an IL-6R by specific point modifications to improve expression and folding. This was achieved by mutations of Cys174→Ser and Cys192→Ser, in the first domain, with or without mutations of Cys258 in the second domain.

In the first scaffold produced, containing IL-6R loops, the expressed scaffold was isolated by low pH extraction with a citrate buffer. The supernatant was purified by HPLC, collecting the monomer and dimer peaks, separately. The retention times of the monomer and dimer were consistent with expected retention times for these size of molecules. Each peak, when purified, was found to have functional activity as measured using ELISA assays and BIAcore microarrays with the ligand. IL-6 bound to the microtitre plates of the biochip respectively. The results for the association and dissociation constants were indicative of published rates for receptors and their ligands. Furthermore the protein peaks did not bind prolactin ligand, demonstrating that the receptor scaffold maintained its specificity to its ligand.

Examples 1 to 7 also demonstrate the methodology to produce a scaffold library based on IL-6R. This was achieved by introduction of random amino acids in the loop regions through PCR and degenerate codon usage. The repertoire was displayed by construction of a phage display library using a pHFAsacII vector. Individual random clones were isolated. Human target antigens were immobilised onto the surface of magnetic beads using standard amine coupling chemistry. After three rounds of phage panning, isolating binders from each round, the phage pools were then assayed for functional activity using ELISA and BIAcore techniques. Each isolate was also sequenced to determine the DNA sequence.

Having produced a simple scaffold, loop grafting was performed, replacing the IL-6R loops with loops from the prolactin receptor. Successful loop grafting was verified by HPLC, which also showed monomer and dimer protein peaks, which, when purified, were found to contain functional activity. Activity was measured using ELISA assays and BIAcore microarrays, with the IL-6 ligand being bound to the microtitre plates of the biochip. The protein peaks were found to bind prolactin and lactogen as expected. In addition, they also bound IL-6. The modified proteins did however, not bind human growth hormone. This result demonstrates that an altered binding profile can be achieved through loop grafting.

### EXAMPLE 9

### Design of a Prolactin Framework

The CBD of human prolactin receptor has the following amino acid sequence:

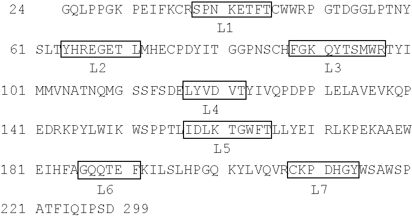

The first FnIII-like domain is defined by amino acids Glu24 to Val125 and the second FnIII like domain by Gln126 to Asp229. Loops L1 to L7 are indicated as boxed residues on the above sequence.

### Modifications

A synthetic gene was designed on the basis of the amino acid listings above, expect with some modifications. In order to improve secretion, several changes were made to the gene construct. Lys30 was changed to Glu. Lysine or arginine charged residues within the first 10 amino acids at the N-terminus prevents the pelB secretion signal from working in the chosen expression system. Arg143Lys144 was changed to GlySer to remove the possibility of providing a proteolytic cleavage site and to provide a restriction enzyme site and a flexible replacement.

The gene was engineered to include convenient restriction sites for mutagensis and bacterial preferred codon usage for high level expression. In particular, the leucine and proline residues are changed.

In order to provide a scaffold library, any of the amino acids within any of the loops may be modified by using degenerate oligonucleotides to generate a diverse set of novel binding moieties as described in Example 7. In this case, the library will consist of a prolactin scaffold with a wide range of different amino acid loop compositions.

Single clones may be isolated from this library and their DNA sequenced to confirm the library diversity.

### EXAMPLE 10

### Design of a IL-11R Scaffold

The CBD of IL-11R has the following amino acid sequence:

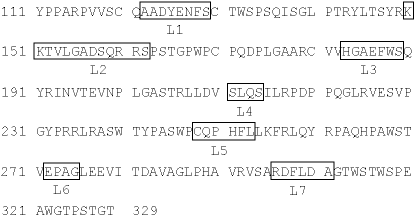

The first FnIII-like domain is defined by amino acids 112-214 and the second FnIII-like domain by amino acids 218-318. Loops L1 to L7 are indicated as boxed residues on the above sequence.

### Modifications.

In the IL-11R framework, the charged Arg115 may be replaced by Glu in order to improve expression in bacterial expression systems using secretion signals, e.g. PelB.

### EXAMPLE 11

### Multidomain Scaffolds

A scaffold consisting of the first FnIII-like domain derived from prolactin and the second FnIII-like domain derived from a human granulocyte colony stimulating factor receptor (G-CSFR) may be constructed.

The first FnIII-liek domain derived from the CBD of prolactin receptor is defined by residues 24-125 [IS THIS CORRECT—see Ex 9 questions] as in Example 9.

The CBD of GSCFR has the following amino acid sequence:

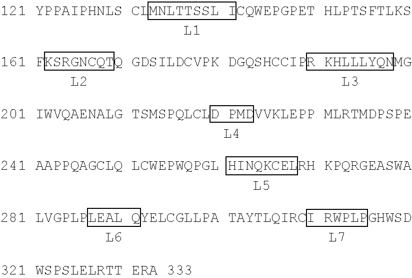

Loops L1 to L7 of the CBD of GCSFR are indicated as boxed residues on the above sequence. The second region of the CBD of GCSFR is defined by residues 237-330.

### Modifications.

In the G-CSFR framework there are several more cysteine residues in addition to the four conserved residues that form two disulphide bonds. Replacement of one or more of Cys186, Cys218, Cys248, Cys252 and Cys295 may therefore be necessary to provide expression of soluble proteins. In the first domain of the prolactin receptor, Lys30 can be changed to Glu as described above in Example 9.

A synthetic gene for the first domain of prolactin receptor and the second domain of GCSFR can be designed with convenient restriction sites and preferred codons as in previous examples. The gene can then be assembled into pHFAsacII phagemid vectors or ribosome display vectors. Phage can be produced and purified from bacterial cells transformed with phagemid using helper phage. Successful display of the scaffold can be confirmed by ELISA using specific targets.

Other modifications can also be made to the scaffold structure described herein, as will be evident to the skilled person. For example, loop L3 of the GCSFR can be extended (i.e. made longer) to form a ‘protruding finger loop’ by inserting extra amino acids. For example, an additional 5 residues can be inserted, either as a predetermined sequence (e.g. AYPPY) or as a random plurality of sequences encoded by a random mixture of 15-mer polynucleotides. Loop 3 can also be made shorter or deleted altogether to provide a possibly a smaller hinge area, and thereby provide a more restrained surface exposed scaffold. Similarly, any other loop in the CBD scaffold can be modified individually or collectively using similar designs to loop 4 as described above.

Other potential modifications include inserting amino acids in areas not specifically associated with the loop region. such as in the hinge region or the domain interface.

### EXAMPLE 12

### Multivalent and Multispecific Scaffolds

It is possible to form multivalent and multispecific scaffolds by either genetic or chemical linkage of two modified cytokine binding domains of the invention. Both linkage formats can result in either covalent or non-covalent bonds or a combination of covalent and non-covalent bonds to effect the association of two or more cytokine binding domains. It will be evident to the skilled person that single cytokine binding domains, or the multivalent or multispecific formats can be genetically or chemically linked to plurality of molecules or linked to a variety of surfaces.

All publications mentioned in the above specification are herein incorporated by reference. Various modifications and variations of the described methods and system of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are readily apparent to those skilled in molecular biology or related fields are intended to be within the scope of the invention.

1.-88. (canceled)

89. A binding moiety comprising an extracellular cytokine binding domain consisting of a first FnIII-like domain and a second FnIII-like domain, wherein the cytokine binding domain comprises a modification which alters at least one property of the cytokine binding domain.

90. The binding moiety according to claim 89, wherein the first and second FnIII-like domains are derived from the extracellular cytokine binding domains from separate sources.

91. The binding moiety according to claim 89, wherein the first and/or second FnIII-like domain(s) is/are derived from the extracellular domain of a receptor selected from the group consisting of IL-2 receptor, IL-3 receptor, IL-4 receptor, IL-5 receptor, IL-6 receptor, IL-7 receptor, IL-9 receptor, IL-11 receptor, IL-12 receptor, IL-13 receptor, IL-15 receptor and IL-21 receptor, G-CSF receptor, GM-CSF receptor, LIF receptor, oncostatin M receptor, cardiotrophin CT-1 receptor, ciliary neutrotrophic factor (CNTF) receptor, prolactin receptor, leptin receptor, erythropoietin receptor, growth hormone receptor, cytokine receptor-like factor 1, class 1 cytokine receptor, thymic stromal lymphopoietin protein receptor or gp130.

92. The binding moiety according to claim 89, wherein at least one loop of the cytokine binding domain is modified such that, as compared with the corresponding loop in the unmodified cytokine binding domain;
(i) the size or area, or both, of the loop is modified; or
(ii) the size of the loop is increased or reduced by at least two amino acid residues; or
(iii) the size of the loop is increased by at least 10 amino acid residues; or
(iv) the size of the loop is increased by up to 20 amino acid residues.

93. The binding moiety according to claim 92, wherein the at least one loop is in the binding interface of the FnIII-like domain.

94. The binding moiety according to claim 89, wherein one or more of intra-domain disulphide-bond forming cysteine residues in the cytokine binding domain is/are modified

95. The binding moiety according to claim 89, wherein the solubility of modified binding moiety is improved.

96. The binding moiety according to claim 95, wherein the solubility of the binding moiety is improved by removing or replacing, or both, disulphide-bond forming cysteine residues within the cytokine binding domain.

97. The binding moiety according to claim 89, wherein the affinity of the modified cytokine binding domain for at least one natural ligand of the unmodified cytokine binding domain is reduced or abolished or the binding specificity of the modified cytokine binding domain is different to that of the unmodified cytokine binding domain, or both.

98. The binding moiety according to claim 97, wherein the unmodified cytokine binding domain is derived from the extracellular domain of a first receptor having specificity for a first ligand, one or more loops of the unmodified cytokine binding domain have been replaced with the corresponding loops of a second receptor having specificity for a second ligand, and the modified cytokine binding domain has specificity for the second ligand.

99. The binding moiety according to claim 98, wherein the first receptor is IL-6 receptor and the second receptor is selected from the group consisting of prolactin receptor, LIF receptor and oncostatin M receptor.

100. The binding moiety according to claim 89 linked to one or more diagnostic reagent(s) or therapeutic agent(s).

101. A pharmaceutical composition comprising a binding moiety according to claim 89 and a pharmaceutically acceptable carrier or diluent.

102. A polynucleotide library comprising a plurality of polynucleotides encoding binding moieties comprising a cytokine binding domain, which polynucleotides comprise one or more modifications in the cytokine binding domain.

103. A method of selecting a binding moiety with an affinity for a target molecule which comprises
(i) providing a plurality of polynucleotides according to claim 102;
(ii) expressing the binding moieties encoded by the polynucleotides; and
(iii) selecting one or more binding moieties having an affinity for the target molecule.

104. The method according to claim 103, wherein the modification(s) is/are in the loop(s) of the cytokine binding domain.

105. The method according to claim 103, wherein the plurality of nucleotides are generated by synthesising a plurality of random synthetic oligonucleotides and inserting the oligonucleotides into a sequence encoding the binding moiety.

106. A method according to claim 103, wherein the target molecule is a cytokine receptor ligand.


SEQUENCE LISTING (88 sequences)

SEQ ID NO: 1 - protein, 209 aa, Mus musculus
YPPASPSNLSCLMHLTTNSLVCQWEPGPETHLPTSFILKSFRSRADCQYQGDTIPDCVAKKRQNNCSIPRKNLLLYQYMAIWVQAENMLGSSESPKLCLDPMDVVKLEPPMLQALDIGPDVVGCLWLSWKPWKPSEYMEQECELRYQPQLKGANWTLVFHLPSSKDQFELCGLHQAPVYTLQMRCIRSSLPGFWSPWSPGLQLRPTMKA

SEQ ID NO: 2 - protein, 210 aa, Homo sapiens
YPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPGCLQLCWEPWQPGLHINQKCELRHKPQRGEASWALVGPLPLEALQYELCGLLPATAYTLQIRCIRWPLPGHWSDWSPSLELRTTERA

SEQ ID NO: 3 - protein, 215 aa, Homo sapiens
ETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPG

SEQ ID NO: 4 - protein, 214 aa, Mus musculus
ETVPLKTLQCYNDYTNHIICSWADTEDAQGLINMTLYHQLEKKQPVSCELSEKLMWSECPSSHRCVPRRCVIPYTRFSITNEDYYSFRPDSDLGIQLMVPLAQNVQPPLPKNVSISSSEDRFLLEWSVSLGDAQVSWLSSKDIEFEVAYKRLQDSWEDAYSLHTSKFQVNFEPKLFLPNSIYAPRVRTRLYPGSSLSGRPSRWSPEAHWDSQPG

SEQ ID NO: 5 - protein, 215 aa, Mus musculus
ETVPLKTLECYNDYTNRIICSWADTEDAQGLINMTLLYHQLDKIQSVSCELSEKLMWSECPSSHRCVPRRCVIPYTRFSNGDNDYYSFQPDRDLGIQLMVPLAQHVQPPPPKDIHISPSGDHFLLEWSVSLGDSQVSWLSSKDIEFEVAYKRLQDSWEDASSLHTSNFQVNLEPKLFLPNSIYAARVRTRLSAGSSLSGRPSRWSPEVHWDSQPG

SEQ ID NO: 6 - protein, 200 aa, Homo sapiens
GDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESV

SEQ ID NO: 7 - protein, 200 aa, Mus musculus
GDKAQPQNLQCFFDGIQSLHCSWEVWTQTTGSVSFGLFYRPSPVAPEEKCSPVVKEPPGASVYTRYHCSLPVPEPSAHSQYTVSVKHLEQGKFIMSYNHIQMEPPTLNLTKNRDSYSLHWETQKMAYSFIEHTFQVQYKKKSDSWEDSKTENLDRAHSMDLSQLEPDTSYCARVRVKPISNYDGIWSKWSEEYTWKTDWV

SEQ ID NO: 8 - protein, 198 aa, Mus musculus
GDKAQPQNLQCFFDGIQSLHCSWEVWTQTTGSVSFGLFYRPSPAAPEEKCSPVVKEPQASVYTRYRCSLPVPEPSAHSQYTVSVKHLEQGKFIMSYYHIQMEPPILNQTKNRDSYSLHWETQKIPKYIDHTFQVQYKKKSESWKDSKTENLGRVNSMDLPQLEPDTSYCARVRVKPISDYDGIWSEWSNEYTWTTDWV

SEQ ID NO: 9 - protein, 202 aa, Homo sapiens
LPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDR

SEQ ID NO: 10 - protein, 200 aa, Mus musculus
FPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDR

SEQ ID NO: 11 - protein, 213 aa, Homo sapiens
NSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLADIQVRWEAPRNADIQKGWMVLEYELQYKEVNETKWKMMDPILTTSVPVYSLKVDKEYEVRVRSKQRNSGNYGEFSEVLYVTLPQMSQFTCEED

SEQ ID NO: 12 - protein, 222 aa, Mus musculus
SSSGKPRFTKCRSPELETFSCYWTEGDNPDLKTPGSIQLYYAKRESQRQAARIAHEWTQEWKECPDYVSAGKNSCYFNSSYTSIWIPYCIKLTTNGDLLDQKCFTVDEIVQPDPPIGLNWTLLNISLGDIQVSWQPPPNADVLKGWIILEYEIQYKEVNESKWKVMGPIWLTYCPVYSLRMDKEHEVRVRSRQRSFEKYSEFSEVLRVIFPQTNILEACEED

SEQ ID NO: 13 - protein, 205 aa, Homo sapiens
EPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKRQVEVSWEYPDTWSTPHSYFSLTFCVQVQGKSKREKKDRVFTDKTSATVICRKNASISVRAQDRYYSSSWSEWASVPCS

SEQ ID NO: 14 - protein, 214 aa, Mus musculus
NFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVKCKGGNVCVQAQDRYYNSSCSKWACVPCR

SEQ ID NO: 15 - protein, 209 aa, Homo sapiens
AALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSD

SEQ ID NO: 16 - protein, 208 aa, Mus musculus
AALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASD

SEQ ID NO: 17 - protein, 205 aa, Homo sapiens
VPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNRWLSVTWQDPHSWNSSFYRLRFELRYRAERSKTFTTWMVKDLQHHCVIHDAWSGLRHVVQLRAQEEFGQGEWSEWSPEAMGTPWTE

SEQ ID NO: 18 - protein, 205 aa, Mus musculus
VPPEEPKLSCFRKNPLVNAICEWRPSSTPSPTTKAVLFAKKINTTNGKSDFQVPCQYSQQLKSFSCQVEILEGDKVYHIVSLCVANSVGSKSSHNEAFHSLKMVQPDPPANLVVSAIPGRRWLKVSWQHPETWDPSYYLLQFQLRYRPVWSKEFTVLLLPVAQYQCVIHDALRGVKHVVQVRGKEELDLGQWSEWSPEVTGTPWI

SEQ ID NO: 19 - protein, 201 aa, Homo sapiens
GNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNS

SEQ ID NO: 20 - protein, 202 aa, Mus musculus
GSIKVLGEPTCFSDYIRTSTCEWFLDSAVDCSSQLCLHYRLMFFEFSENLTCIPRNSASTVCVCHMEMNRPVQSDRYQMELWAEHRQLWQGSFSPSGNVKPLAPDNLTLHTNVSDEWLLTWNNLYPSNNLLYKDLISMVNISREDNPAEFIVYNVTYKEPRLSFPINILMSGVYYTARVRVRSQILTGTWSEWSPSITWYNH

SEQ ID NO: 21 - protein, 204 aa, Homo sapiens
GQLPPGKPEEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEPYLWIKWSPPTLIDLKTGWFTLLYEIRLKPEKAAEWEIHFAGQQTEFKILSLHPGQKYLVQVRCKPDHGYWSAWSPATFIQIPSD

SEQ ID NO: 22 - protein, 203 aa, Mus musculus
GQSPPGKPEIHKCRSPDKETFTCWWNPGSDGGLPTNYSLTYSKEGEKNTYECPDYKTSGPNSCFFSKQYTSIWKIYIITVNATNEMGSSTSDPLYVDVTYIVEPEPPRNLTLEVKQLKTYLWVKWLPPTITDVKTGWFTMEYEIRLKSEEADEWEIHFTGHQTQFKVFDLYPGQKYLVQTRCKPDHGYWSRWGQEKSIEIPND

SEQ ID NO: 23 - protein, 208 aa, Homo sapiens
LPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGDQLSVRWVSPPALKDFLFQAKYQIRYRVEDSVDWKVVDDVSNQTSCRLAGLKPGTVYFVQVRCNPFGIYGSKKAGIWSEWSHPTAASTPRSE

SEQ ID NO: 24 - protein, 208 aa, Mus musculus
LPPEKPFNISCWSRNMKDLTCRWTPGAHGETFLHTNYSLKYKLRWYGQDNTCEEYHTVGPHSCHIPKDLALFTPYEIWVEATNRLGSARSDVLTLDVLDVVTTDPPPDVHVSRVGGDQLSVRWVSPPALKDFLFQAKYQIRYRVEDSVDWKVVDDVSNQTSCRLAGLKPGTVYFVQVRCNPFGIYGSKKAGIWSEWSHPTAASTPRSE

SEQ ID NO: 25 - protein, 198 aa, Homo sapiens
VAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASSRCTLYWRDEGLVLLNRLRYRPSNSRLWNMVNVTKAKGRHDLLDLKPFTEYEFQISSKLHLYKGSWSDWSESLRAQTPEEE

SEQ ID NO: 26 - protein, 201 aa, Mus musculus
VAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASSRGTLQWEDEGQVVLNQLRYQPLNSTSWNMVNATNAKGKYDLRDLRPFTEYEFQISSKLHLSGGSWSNWSESLRTRTPEEE

SEQ ID NO: 27 - protein, 208 aa, Homo sapiens
YPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTG

SEQ ID NO: 28 - protein, 208 aa, Mus musculus
FPPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDAVAGLPHAVRVSARDFLDAGTWSAWSPEAWGTPSTG

SEQ ID NO: 29 - protein, 208 aa, Mus musculus
FPPARPEVSCQAVDYENFSCTWSPGQVSGLPTRYLTSYRKKTLPGAESQRESPSTGPWPCPQDPLEASRCVVHGAEFWSEYRINVTEVNSLGASTCLLDVRLQSILRPDPPQGLRVESVPGYPRRLHASWTYPASWRRQPHFLLKFRLQYRPAQHPAWSTVEPIGLEEVITDTVAGLPHAVRVSARDFLDAGTWSAWSPEAWGTPSTG

SEQ ID NO: 30 - protein, 208 aa, Homo sapiens
LPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQA

SEQ ID NO: 31 - protein, 207 aa, Mus musculus
LPPREPVLSCRSNTYPKGFYCSWHLPTPTYIPNTFNVTVLHGSKIMVCEKDPALKNRCHIRYMHLFSTIKYKVSISVSNALGHNTTAITFDEFTIVKPDPPENVVARPVSNRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQA

SEQ ID NO: 32 - protein, 207 aa, Homo sapiens
GPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 33 - protein, 206 aa, Mus musculus
ALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 34 - protein, 259 aa, Homo sapiens
DVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPETSDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAV

SEQ ID NO: 35 - protein, 251 aa, Mus musculus
DVFLLALGTEPLNCFSQTFEDLTCFWDEEEAAPSGTYQLLYAYRGEKPRACPLYSQSVPTFGTRYVCQFPAQDEVRLFFPLHLWVKNVSLNQTLIQRVLFVDSVGLPAPPRVIKARGGSQPGELQIHWEAPAPEISDFLRHELRYGPTDSSNATAPSVIQLLSTETCCPTLWMPNPVPVLDQPPCVHPTASQPHGPAPFLTVKGGSCLVSGLQASKSYWLQLRSQPDGVSLRGSWGPWSFPVTVDLPGDAV

SEQ ID NO: 36 - protein, 209 aa, Homo sapiens
DVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINGLLKISWEKPVFPENNLQFQIRYGLSGKEVQWKMYEVYDAKSKSVSLPVPDLCAVYAVQVRCKRIDGLSYMSNWSNPAYTVVMDIKV

SEQ ID NO: 37 - protein, 210 aa, Mus musculus
DVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKV

SEQ ID NO: 38 - protein, 209 aa, Homo sapiens
DANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISMSSPPLVPFPLQYQVKYSENSTTVIREADKIVSATSLLVDSILPGSSYEVQVRGKRLDGPGIWSDWSTPRVFTTQD

SEQ ID NO: 39 - protein, 207 aa, Mus musculus
GVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKLSWDSQTMAPFPLQYQVKYLENSTIVREAAEIVSATSLIVDSVLPGSSYEVQVRSKRLDGSGVWSDWSSPQVFTTQD

SEQ ID NO: 40 - protein, 209 aa, Homo sapiens
GWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYRNRGDPQAVSPRRKLISVDSRSVSLLPLEFRKDSSYELQVRAGPMPGSSYQGTWSEWSDPVIFQTQSEE

SEQ ID NO: 41 - protein, 213 aa, Mus musculus
AWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYSEPSNYVLEGKLQYELQYRNLRDPYAVRPVTKLISVDSRNVSLLPEEFHKDSSYQLQVRAAPQPGTSFRGTWSEWSDPVIFQTQAGE

SEQ ID NO: 42 - protein, 206 aa, Homo sapiens
VALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETA

SEQ ID NO: 43 - protein, 205 aa, Mus musculus
VTIGLQCFTLDLKMVTCQWQQQDRTSSQGFFRHSRTRCCPTDRDPTWEKCEEEEPRPGSQPALVSRCHFKSRNDSVIHILVEVTTAQGAVHSYLGSPFWIHQAVLLPTPSLHWREVSSGRLELEWQHQSSNAAQETCYQLRYTGEGHQDWKVLEPSLGARGGTLELRPRARYSLQLRARLNGPTYQGPWSAWSPPARVSTGSETA

SEQ ID NO: 44 - protein, 198 aa, Homo sapiens
GSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDLEATVHWAPPTWPSHKVLICQFHYRRCQEAAWTLLEPELKTIPLTPVEIQDLELATGYKVYGRCRMEKEEDLWGEWSPILSFQTPPSA

SEQ ID NO: 45 - protein, 198 aa, Mus musculus
GSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLD

SEQ ID NO: 46 - protein, 206 aa, Homo sapiens
GTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAD

SEQ ID NO: 47 - protein, 206 aa, Mus musculus
NCSHECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPAD

SEQ ID NO: 48 - protein, 215 aa, Homo sapiens
GPRSRTFTCLTNNILRIDCHWSAPELGQGSSPWLLFTSNQAPGGTHKCILRGSECTVVLPPEAVLVPSDNFTITFHHCMSGREQVSLVDPEYLPPRHVKLDPPSDLQSNISSGHCILTWSISPALEPMFTLLSYELAFKKQEEAWEQAQHRDHIVGVTWLILEAFELDPGFIHEARLRVQMATLEDDVVEEERYTGCQWSEWSQPVCFQAPQRQG

SEQ ID NO: 49 - protein, 212 aa, Mus musculus
GQKAGAFTCLSNSIYRIDCHWSAPELGQESRAWLLFTSNQVTEIKHKCTFWDSMCTLVLPKEEVFLPFDNFTITLHRCIMGQEQVSLVDSQYLPRRHIKLDPPSDLQSNVSSGRCVLTWGINLALEPLITSLSYELAFKRQEEAWEARHDRIVGVTWLILEAVELNPGSIYEARLRVQMTLESYKDKTEGEYYKSHWSEWSQPVSFPQRRQG

SEQ ID NO: 50 - protein, 193 aa, Homo sapiens
GSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 51 - protein, 214 aa, Mus musculus
GSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHGQLRMDWNVSEEAGAEVQFRRRMPTTNWTLGDCGPQVNSGSGVLGDICGSMSESCLCPSENMAQEIQIRRRRRLSSGARGGPWSEWSMPVCVPPE

SEQ ID NO: 52 - protein, 215 aa, Homo sapiens
GDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTNLTVRIRVKTNKLCYEDDKLWSNWSQEMSIG

SEQ ID NO: 53 - protein, 215 aa, Mus musculus
GDPESAVTELKCIWHNLSYMKCSWLPGRNTSPDTHYTLYYWYSSLEKSRQCENIYREGQHIACFSKLTKVEPSFEHQNVQIMVKDNAGKIRPSCKIVSLTSYVKPDPPHIKHLLLKNGALLVQWKNPQNFRSRCLTYEVEVNNTQTDRHNILEVEEDKCQNSESDRNMEGTSCFQLPGVLADAVYTVRVRVWVKTNKLCFDDNKLWSDWSEAQSI

SEQ ID NO: 54 - protein, 197 aa, Homo sapiens
GIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFIEIREDDTTLVTATVENETYTLKTTNETRQLCFVVRSKVNIYCSDDGIWSEWSDKQCWEG

SEQ ID NO: 55 - protein, 200 aa, Mus musculus
GSLETKIQDMKCIYYNWQYLVCSWKPGKTVYSDTNYTMFFWYEGLDHALQCADYLQHDEKNVGCKLSNLDSSDYKDFFICVNGSSKLEPIRSSYTVFQLQNIVKPLPPEFLHISVENSIDIRMKWSTPGGPIPPRCYTYEIVIREDDISWESATDKNDMKLKRRANESEDLCFFVRCKVNIYCADDGIWSEWSEEECWEG

SEQ ID NO: 56 - protein, 210 aa, Homo sapiens
GSPGTSIVNLTCTTNTTEDNYSRLRSYQVSLHCTWLVGTDAPEDTQYFLYYRYGSWTEECQEYSKDTLGRNIACWFPRTFILSKGRDWLSVLVNGSSKHSAIRPFDQLFALHAIDQINPPLNVTAEIEGTRLSIQWEKPVSAFPPHCFDYEVKIHNTRNGYLQIEKLMTNAFISIIDDLSKYDVQVRAAVSSMCREAGLWSEWSQPIYVG

SEQ ID NO: 57 - protein, 211 aa, Mus musculus
GSPGTSVTNLTCTTHTVVSSHTHLRPYQVSLRCTWLVGKDAPEDTQYFLYYRFGVLTEKCQEYSRDALNRNTACWFPRTFINKSKGFEQLAVHINGSSKRAAIKPFDQLFSPLAIDQVNPPRNVTVEIESNSLYIQWEKPLSAFPDHCFNELRKIYNTKNGHIQKEKLIANKFISKIDDVSTYSIQVRAAVSSPCRMPGRWGEWSQPIYVG

SEQ ID NO: 58 - protein, 218 aa, Homo sapiens
GREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQQLDVHRKNTQPGTENLLINVSGDLENRTNEPSSEPRAKHSVKIRAADVRILNWSSWSEAIEFGSDDGNLGSVYIYVLL

SEQ ID NO: 59 - protein, 213 aa, Mus musculus
ESAAGSGAENLTCEIRAARFLSCAWREGPAAPADVRYSLRVLNSTGHDVARCMADPGDDVITQCIANDLSLLGSEAYLVVTGRSGAGPVRFLDDVVATKALERLGPPRDVTASCNSSHCTVSWAPPSTKASLTARDFQFEVQWQSAEPGSTPRKVLVVEETRLAFPSPAPHGGHKVKVRAGDTRMKHWGEWSPAHPLEAEDTRVPGALLYAVT

SEQ ID NO: 60 - protein, 193 aa, Homo sapiens
SGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHENRKFRYELQIQKRMQPVITEQVRDRTSFQLLNPGTYTVQIRARERVYEFLSAWSTPQRFE

SEQ ID NO: 61 - protein, 210 aa, Mus musculus
DGDHEAAAQDLRCWVHEGQLSCQWERGPKATGDVHYRMFWRDVRLGPAHNRECPHYHSLDVNTAGPAPHGGHEGCTLDLDTVLGSTPNSPDLVPQVTITVNGSGRAGPVPCMDNTVDLQRAEVLAPPTLTVECNGSEAHARWVARNEFHHGLLGYTLQVNQSSRSEPQEYNVSIPHEWVPNAGAISFRVKSRSEVYPRKLSSWSEAWGLV

SEQ ID NO: 62 - protein, 213 aa, Homo sapiens
DFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTWDHSTEQSVDYRHKFSLPSVDGQKRYTFRVRSRFNPLCGSAQHWSEWSHPIHWGSNTSKE

SEQ ID NO: 63 - protein, 216 aa, Mus musculus
DLILTSTAPEHLSAPTLPLPEVQCFVFNIEYMNCTWNSSSEPQATNLTLHRYRKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEE

SEQ ID NO: 64 - protein, 187 aa, Homo sapiens
VQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRD

SEQ ID NO: 65 - protein, 191 aa, Mus musculus
GDVTVVCHDLETVEVTWGSGPDHHSANLSLEFRYGTGALQPCPRYFLSGAGVTSGCILPAARAGLLELALRDGGGAMVFKARQRASAWLKPRPPWNVTLLWTPDGDVTVSWPAHSYLGLDYEVQHRESNDDEDAWQTTSGPCCDLTVGGLDPARCYDFRVRASPRAAHYGLEAQPSEWPAVTRLSGAASAG

SEQ ID NO: 66 - protein, 200 aa, Homo sapiens
GAPHDLKCVTNNLQVWNCSWKAPSGTGRGTDYEVCIENRSRSCYQLEKTSIKIPALSHGDYEITINSLHDFGSSTSKFTLNEQNVSLIPDTPEILNLSADFSTSTLYLRWNDRGSVPPHRSNVIWEIKVLRKESMELVKLVTHNTTLNGKDTLHHWSWASDMPLEACAIHFVEIRCYIDNLHFSGLEEWSDWSPVKNISW

SEQ ID NO: 67 - protein, 195 aa, Mus musculus
GVQDLKCTTNNMRVWDCTWPAPLGVSPGTVKDICIKDRFHSCHPLETTNVKIPALSPGDHEVTINYLNGFQSKFTLNEKDVSLIPETPEILDLSADFFTSSELLKWNDRGSALPHPSNATWEIKVLQNPRTEPVALVLLNTMLSGKDTVQHWNWTSDLPLQCATHSVSLRWHIDSPHFSGYKEWSPWSPLKNISW

SEQ ID NO: 68 - protein, 204 aa, Homo sapiens
YPPDTPQQLNCETHDLKEIICSWNPGRVTALVGPRATSYTLVESFSGKYVRLKRAEAPTNESYQLLFQMLPNQEIYNFTLNAHNPLGRSQSTILVNITEKVYPHTPTSFKVKDINSTAVKLSWHLPGNFAKINFLCEIKIKKSNSVQEQRNVTIKGVENSSYLVALDKLNPYTLYTFRIRCSTETFWKWSKWSNKKQHLTTEAS

SEQ ID NO: 69 - protein, 204 aa, Mus musculus
YPPDVPQKLSCETHDLKEIICSWNPGRITGLVGPRNTEYTLFESISGKSAVFHRIEGLTNETYRLGVQMHPGQEIHNFTLTGRNPLGQAQSAVVINVTERVAPHDPTSLKVKDINSTVVTFSWYLPGNFTKINLLCQIEICKANSKKEVRNATIRGAEDSTYHVAVDKLNPYTAYTFRVRCSSKTFWKWSRWSDEKRHLTTEAT

SEQ ID NO: 70 - protein, 116 aa, Homo sapiens
VLAERLPLTPVSLKSVTNSTRQSLHLQWTVENLPYHQELKMVFQIQISRIETSNVIWVGNYSTTVKWNQVLHWSWESELPLECATHFVRIRSLVDDAKFPEPNFWSNWSSWEEVSV

SEQ ID NO: 71 - protein, 115 aa, Mus musculus
VLEEPLPTLPEIHKVSVQLKLQEVNLEWTVPALTHEELNMIFQIEISRLNISNTIWVENYSTTVKREEAVRWNWTSDIPLECVKHFIRIWALVDDTKSLPQSSWGNWSSWKEVNA

SEQ ID NO: 72 - protein, 195 aa, Homo sapiens
KVLEEPKDFSCETEDFKTLHCTWDPGTDTALGWSKQPSQSYTLFESFSGEKKLCTHKNWCNWQITQDSQETYNFTLIAENYLRKRSVNILFNLTHRVYLMNPFSVNFENVNATNAIMTWKVHSIRNNFTYLCQIELHGEGKMMQYNVSIKVNGEYFLSELEPATEYMARVRCADASHFWKWSEWSGQNFTTLEAA

SEQ ID NO: 73 - protein, 195 aa, Mus musculus
KVLEEPKNVSCETRDFKTLDCSWEPGVDTTLTWRKQRFQNYTLCESFSKRCEVSNYRNSYTWQITEGSQEMYNFTLTAENQLRKRSVNINFNLTHRVHPKAPQDVTLKIIGATKANMTWKVHSHGNNYTYLCQCKLQYGEVIHEHNVSVHMSANYLFSDLDPDTKYKAFVRCASANHFWKWSDWTQKEFSTPETA

SEQ ID NO: 74 - protein, 206 aa, Homo sapiens
GDLEDAELDDYSFSCYSQLEVNGSQHSLTCAFEDPDVNTTNLEFEICGALVEVKCLNFRKLQEIYFIETKKFLLIGKSNICVKVGEKSLTCKKIDLTTIVKPEAPFDLSVIYREGANDFVVTFNTSHLQKKYVKVLMHDVAYRQEKDENKWTHVNLSSTKLTLLQRKLQPAAMYEIKVRSIPDHYFKGFWSPSYYERTPEINNSSG

SEQ ID NO: 75 - protein, 209 aa, Mus musculus
GDLEDADADDHSFWCHSQLEVDGSQHLLTCAFNDSDINTANLEFQICGALLRVKCLTLNKLQDIYFIKTSEFLLIGSSNICVKLGQKNLTCKNMAINTIVKAEAPSDLKVVYRKEANDFLVTFNAPHLKKKYLKKVKHDVAYRPARGESNWTHVSLFHTRTTIPQRKLRPKAMYEIKVRSIPHNDYFKGFWSEWSPSSTFETPEPKNQG

SEQ ID NO: 76 - protein, 216 aa, Drosophila melanogaster
KSKVYVGTRPLLVRDFNCLDYDFQFMVCNFTQPPNTVITKYNISYNTNNDWRYSNTLDCNFDSAPVVTCNLTDDNYKRFSETFYFRLSISNALGHETQPITINHFERLVPARPGQNLTLLNRTESSVCLSWEMPRRSNYNRGLVWQVRVTPQNFEPITRPSWRNHTLTIKDTLCLTELPFAGYNYTLRVRVRANQNNTLWSEPMIYAFATAPAPPR

SEQ ID NO: 77 - protein, 216 aa, Drosophila melanogaster
QSHVCVRVYALLNLKDFVRCDVVYYLRCTFSRMENGNFENKTHYQLAMGRAKPIDCRKSEDERSRGKVECSVPIDPNSRAPEWRDFRLIMSDDLGNQSKVLRLTQAEMEVLEWPRGKHNMIQTPNQTCLEWNGPFIYPNRTFELNVQFRHSKLPNLSRNLTVSQMRAVSVFDQVGFGNPPEGNQLFYVSLSRRLHGSPWSERYPEFKLTTNASLPA

SEQ ID NO: 78 - protein, 204 aa, Artificial
  Consensus
GPPEKPAQNLSCFTDNLETLTCSWEPGPDTGLPTNYTLFYRLSSLDKIKECPLYLSAGLGRSRCHIPDDLSSTSPYTVSVTATNPLGSSSSSDLTFDLTDIVKPDPPLNLTVSISSESGRLKLSWEPPSSWPSYFDLKYEVRYRPENDSWEDWKVVELDSTSFTLSDLEPGTSYEVRVRARPDSGSGTWSEWSPPASFTIPEGE

SEQ ID NO: 79 - protein, 207 aa, Homo sapiens
LPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPWLAPVSSDLKYTLRFRTVNSTSWMEVNFAKNRKDKNQTYNLTGLQPFTEYVIALRCAVKESKFWSDWSQEKMGMTEEEAP

SEQ ID NO: 80 - protein, 194 aa, Mus musculus
LPTKPENISCVFYFDRNLTCTWRPEKETNDTSYIVTLTYSYGKSNYSDNATEASKSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVP

SEQ ID NO: 81 - protein, 197 aa, Mus musculus
HPPDAPSNLTCVIYEYSGNMTCTWNTGKPTYIDTKYIVHVKSLETEEEQQYLASSYVKISTDSLQGSRKYLVWVQAVNSLGMENSQQLHVHLDDIVIPSASIISRAETTNDTVPKTIVYWKSKTMIEKVFCEMRYKTTTNQTWSVKEFDANFTYVQQSEFYLEPDSKYVFQVRCQETGKRNWQPWSSPFVHQTSQEG

SEQ ID NO: 82 - protein, 197 aa, Homo sapiens
YPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETA

SEQ ID NO: 83 - protein, 325 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNIL

SEQ ID NO: 84 - protein, 46 aa, Artificial
  Synthetic
RSKTFTTWMVKDLQHHCVIHDAWSGLRHVVQLRAQEEFGQGEWSEW

SEQ ID NO: 85 - protein, 46 aa, Artificial
  Synthetic
RSKTFTTWAQSRWQHHSVIHDAWSGLRHVVQLRAQEEFGQGEWSEW

SEQ ID NO: 86 - protein, 46 aa, Artificial; identical to SEQ ID NO: 84
  Synthetic

SEQ ID NO: 87 - protein, 46 aa, Artificial
  Synthetic
RSKTFTTWSRQDNQHHSVIHDAWSGLRHVVQLRARNEVRVGEWSEW

SEQ ID NO: 88 - protein, 205 aa, Artificial
  Synthetic
DVPPEEPQLSCFSPNKETFVCEWGPRSTPSLTTKAVLLVHREGETLMFQEPCQYSQESQKFSCHFGKQYTSMWRTYIVSMSVASSVGSKFSDELYVDVTYILQPDPPANITVTAVARNPRWLSVTWQDPHLIDLKTGWFTLRFELRYRAERSKTFTTWFAGQQHHSVIHDAWSGLRHVVQLRAKPDHGYWSEWSPEAMGTPWTES